(19) United States
(12) Patent Application Publication
(10) Pub. No.: US 2010/0298930 A1
(43) Pub. Date: Nov. 25, 2010

(54) ACCESS TO THE LEFT ATRIUM AND REDUCTION OF MITRAL VALVE LEAFLET MOBILITY

(76) Inventor: Boris Orlov, Haifa (IL)

Correspondence Address: MARTIN D. MOYNIHAN d/b/a PRTSI, INC., P.O. BOX 16446, ARLINGTON, VA, 22215, US

(21) Appl. No.: 12/745,700

(22) PCT Filed: Dec. 2, 2008
(86) PCT No.: PCT/IL08/01565

§ 371 Date: Aug. 9, 2010

Related U.S. Application Data
(60) Provisional application No. 60/991,748, filed on Dec. 2, 2007.
(60) Provisional application No. 61/041,942, filed on Apr. 3, 2008.

(51) Int. Cl. A61F 2/24

(52) U.S. Cl. 623/2.11

(57) ABSTRACT

Disclosed are a method for engaging cardiac valve leaflets, including a) directing a distal end of an elongated catheter body of a leaflet-engaging device into a coronary sinus of a heart, b) passing a first leaflet-engaging component located proximate to the distal end of the elongated catheter body of the leaflet-engaging device through cardiac tissue separating the coronary sinus and a left atrium of the heart to enter a left atrium of the heart, and c) engaging a first cardiac valve leaflet with the first leaflet-engaging component, thereby engaging at least one cardiac valve leaflet. Related apparatus and methods are also described.

### RELATED APPLICATION/S

This application claims priority from U.S. Provisional Patent Application No. 60/991,748, filed by the same Inventor on 2 Dec. 2007, and from US Provisional Patent Application No. 61/041,942 filed on 3 Apr. 2008.

The contents of all of the above documents are incorporated by reference as if fully set forth herein.

### FIELD AND BACKGROUND OF THE INVENTION

The present invention, in some embodiments thereof, relates to the fields of cardiac surgery and cardiology. Some embodiments relate to accessing the left atrium or the mitral valve of a mammalian heart through the coronary sinus. Some embodiments relate to the reduction of mobility of one or both mitral valve leaflets, for example to the extent that performance of surgical intervention to the mitral valve, for example, deployment of a leaflet-edge capturing device to perform a “bow-tie” repair of the mitral valve, is relatively quick, simple, and accurate.

The human heart 10, depicted in cross-sectional long axis view in FIG. 1, is a muscular organ that pumps deoxygenated blood through the lungs to oxygenate the blood and pumps oxygenated blood to the rest of the body by rhythmic contractions of four chambers.

After having circulated in the body, deoxygenated blood from the body enters the right atrium 12 through the vena cava 14 (superior vena cava 14a or inferior vena cava 14b). Right atrium 12 contracts, pumping the blood through a tricuspid valve 16 into the right ventricle 18. Right ventricle 18 contracts, pumping the blood through the pulmonary semi-lunar valve 20 into the pulmonary artery 22 which splits to two branches, one for each lung. The blood is oxygenated while passing through the lungs and reenters the heart to the left atrium 24.

Left atrium 24 contracts, pumping the oxygenated blood through the mitral valve 26 into the left ventricle 28. Left ventricle 28 contracts, pumping the oxygenated blood through the aortic semi-lunar valve 30 into the aorta 32. From aorta 32, the oxygenated blood is distributed to the rest of the body.

Physically separating left atrium 24 and right atrium 12 is an interatrial septum 33.

Transversely curving in a groove between left atrium 24 and left ventricle 28, slightly above the plane defined by mitral valve 26 is a coronary sinus 52, a large vein. Deoxygenated blood from the muscles of heart 10 enters into coronary sinus 52 and drains directly into right atrium 12 through a coronary sinus cardiac ostium 54, an opening superior to the septal leaflet of tricuspid valve 16. In adults, a coronary sinus 52 is typically tubular having a diameter of between about 7 mm to 10 mm, although in some pathologies e.g., a coronary sinus adopts a “wind-sock” shape having an increased diameter at cardiac ostium 54, e.g., up to about 12 mm in cases of Atrioventricular Junctional Reentry Tachycardia.

Mitral valve 26, depicted in FIG. 2A (top view) and in FIG. 2B (cross-sectional long axis view) is defined by an approximately circular mitral annulus 34 that defines a mitral lumen 36. Attached to the periphery of mitral annulus 34 is an anterior leaflet 38 and a smaller posterior leaflet 40, leaflets 38 and 40 joined at commissures 41.

The typical area of mitral lumen 36 in a healthy adult is between 4 and 6 cm² while the typical total surface area of leaflets 38 and 40 is approximately 12 cm². Consequently and as depicted in FIG. 2B, leaflets 38 and 40 curve downwards into left ventricle 28 and coapt to accommodate the excess leaflet surface area, producing a coaptation surface 42 that constitutes a seal. The typical length of coaptation surface 42 in a healthy heart 10 of an adult is approximately 7-8 mm.

Anterior leaflet 38 and posterior leaflet 40 are connected to papillary muscles 44 at the bottom of left ventricle 28 by posterior chordae 46 and anterior chordae 48.

During diastole, left atrium 24 contracts to pump blood into left ventricle 28 through mitral valve 26. The blood flows through mitral lumen 36 pushing leaflets 38 and 40 into left ventricle 28 with little resistance.

During systole left ventricle 28 contracts to pump blood into aorta 32 through aortic semi-lunar valve 30. Mitral annulus 34 contracts pushing leaflets 38 and 40 inwards, reducing the area of mitral lumen 36 by about 20% to 30% and increasing the length of coaptation surface 42. The pressure of blood in left ventricle 28 pushes against the ventricular surfaces of leaflets 38 and 40, tightly pressing leaflets 38 40 together at coaptation surface 42 so that a tight leak-proof seal is formed. To prevent prolapse of leaflets 38 and 40 into left atrium 24, papillary muscles 44 contract, pulling the edges of leaflets 38 and 40 into left ventricle 28 through posterior chordae 46 and anterior chordae 48, respectively.

As is clear from the description above, an effective seal of mitral valve 26 is dependent on a sufficient degree of coaptation, in terms of length, area and continuity of coaptation surface 42. If coaptation surface 42 is insufficient or non-existent, there is mitral valve insufficiency, that is, regurgitation of blood from left ventricle 28 into left atrium 24. A lack of sufficient coaptation may be caused by any number of physical anomalies that allow leaflet prolapse (e.g., elongated or ruptured chordae 46 and 48, weak papillary muscles 44) or prevent coaptation (e.g., short chordae 46 and 48, small leaflets 38 and 40).

Mitral valve insufficiency leads to many complications including arrhythmia, atrial fibrillation, cardiac palpitations, chest pain, congestive heart failure, fainting, fatigue, low cardiac output, orthopnea, paroxysmal nocturnal dyspnea, pulmonary edema, shortness of breath, and sudden death.

There are a number of pathologies that lead to a mitral valve insufficiency including collagen vascular disease, ischemic mitral regurgitation, myxomatous degeneration of leaflets 38 and 40 and rheumatic heart disease.

In ischemic mitral regurgitation (resulting, e.g., from myocardial infarction, chronic heart failure, or surgical or catheter revascularization), leaflets 38 and 40 and chordae 46 and 48 have normal structure and the mitral valve insufficiency results from altered geometry of left ventricle 28. As a result of ischemia, portions of the outer walls of left ventricle 28 necrose. During healing, the necrotic tissue is replaced with unorganized tissue leading to remodeling of the heart which reduces coaptation through distortion of mitral annulus 34 and sagging of the outer wall of left ventricle 28 which displaces papillary muscles 44.

In FIGS. 3A (top view) and 3B (cross-sectional long axis view), the reduction of coaptation resulting from ischemia is depicted for a mitral valve 26 of an ischemic heart 50 that has undergone mild remodeling and suffers from ischemic mitral regurgitation. In FIG. 3B is seen how an outer wall of left ventricle 28 sags, displacing papillary muscles 44 which, through chordae 46 and 48, pulls leaflets 38 and 40 apart, reducing coaptation. The incomplete closure of mitral valve 26 is seen in FIGS. 3A and 3B.

Initially, ischemic mitral regurgitation is a minor problem, typically leading only to shortness of breath during physical exercise due to the fact that a small fraction of blood pumped by left ventricle 28 is pumped into left atrium 24 and not into aorta 32, reducing heart capacity. To compensate for the reduced capacity, left ventricle 28 beats harder and consequently remodeling continues. Ultimately leaflet coaptation is entirely eliminated as leaflets 38 and 40 are pulled further and further apart, leading to more blood regurgitation, further increasing the load on left ventricle 28, and further remodeling. Ultimately, the left side of the heart fails and the person dies.

Apart from humans, mammals that suffer from mitral valve insufficiency include horses, cats, dogs, cows, sheep and pigs.

Currently, it is accepted to use open-heart surgical methods to improve mitral valve functioning by many different methods, including: modifying the subvalvular apparatus (e.g. lengthening the chordae) to improve leaflet coaptation; implanting an annuloplasty ring, e.g., as described in U.S. Pat. Nos. 3,656,185; 6,183,512 and 6,250,308 to force mitral valve annulus 34 into a normal shape; or implanting devices in the mitral valve to act as prosthetic leaflets, e.g., United States Patent applications published as US 2002/065554, US 2003/0033009, US 2004/0138745 or US 2005/0038509.

One novel method to improve mitral valve functioning is the “bow-tie” repair described by Alfieri et al. in J. Thorac. Cardiovasc. Surg. 2001, 122, 674-68. As depicted in FIG. 4A, a free edge 56 of an anterior mitral valve leaflet 38 is secured to a free edge 58 of a posterior mitral valve leaflet 40, for example with a suture 60, approximately midway between commissures 41. Such a treated mitral valve 28 becomes a double-orifice valve having an opening with an appearance of a bow-tie, with reduced leaflet motion, preventing regurgitation into the left ventricle 28.

A percutaneous suture leaflet repair system named Mobius is offered by Edwards Lifesciences Inc., of Orange, Calif., USA. The Mobius system involves delivering sutures across the mitral valve leaflets, edge-to-edge, using an Alfieri approach, and securing the sutures with a Nitinol clip.

A number of percutaneous methods for implementing the “bow-tie” repair, generally involving the use of a catheter to deploy a leaflet-joining device, have been disclosed, see for example the PCT patent application published as WO 99/00059; EP patent application EP 1674040 and U.S. Pat. Nos. 6,165,183; 6,575,971; 6,719,767 and 6,752,813. A challenge in implementing any percutaneous method for implementing a “bow-tie” repair of a mitral valve results from leaflet motion: it is difficult to catch both the anterior and the posterior leaflets, properly align the leaflets and then deploy a leaflet-joining device such as suture 60.

Reference is now additionally made to FIG. 4B (prior art), which is a schematic depiction of a transcatheter mitral valve therapy device 203 inserted into the left ventricle 28 through the left atrium 24.

The transcatheter mitral valve therapy device 203 depicted as a typical example of a mitral valve therapy device 203, is a MitraClip™ provided by Evalve Inc., of 4045 Campbell Ave., Menlo Park, Calif. 94025, USA. A catheter 204 is inserted into the left atrium 24. The therapy device 203 is advanced beyond the catheter 204, through the mitral valve 26, into the left ventricle 28. After insertion into the left ventricle 28, the therapy device 203 is retracted somewhat, until touching an anterior leaflet 38 and a posterior leaflet 40 of the mitral valve 26. The anterior leaflet 38 and the posterior leaflet 40 are clamped together by the therapy device 203, producing a result similar to that produced by the suture 60 depicted in FIG. 4A.

The following references provide additional background:

### PCT Published Patent Application WO 99/00059;

### PCT Published Patent Application WO 07/138572;

### PCT Published Patent Application PCT/IL2008/000758;

EP Published Patent Application No. EP 1674040;
U.S. Pat. No. 6,165,183;
U.S. Pat. No. 6,575,971;
U.S. Pat. No. 6,719,767;
U.S. Pat. No. 6,752,813;
U.S. Pat. No. 6,764,510;
U.S. Pat. No. 6,790,231;
U.S. Pat. No. 7,004,958;

### US Published Patent Application 2004/0260393;

### US Published Patent Application 2008/091264; and

### U.S. Provisional Patent Application 60/924,869 of the Inventor.

The contents of all of the above documents are incorporated by reference as if fully set forth herein.

### SUMMARY OF THE INVENTION

The present invention, in some embodiments thereof, relates to the fields of cardiac surgery and cardiology. Some embodiments of the present invention relate to access of the left atrium or the mitral valve of a mammalian heart through the coronary sinus. Some embodiments of the present invention relate to reduction of the mobility of one or both mitral valve leaflets. By way of a non-limiting example, reduction of the mobility of one or both mitral valve leaflets to the extent that performance of surgical intervention of the mitral valve, is made to be relatively quick, simple and accurate.

According to an aspect of some embodiments of the present invention there is provided a method for engaging mitral valve leaflets, including a) directing a distal end of an elongated catheter body of a leaflet-engaging device into a coronary sinus of a heart, b) passing a first leaflet-engaging component located proximate to the distal end of the elongated catheter body of the leaflet-engaging device through cardiac tissue separating the coronary sinus and a left atrium of the heart to enter a left atrium of the heart, and c) engaging a first mitral valve leaflet with the first leaflet-engaging component, thereby engaging at least one mitral valve leaflet.

According to some embodiments of the invention, the heart is a beating heart.

According to some embodiments of the invention, the first leaflet-engaging component engages the cardiac valve leaflet at an atrial face of the first mitral valve leaflet.

According to some embodiments of the invention, prior to the engaging of the first mitral valve leaflet, the first leaflet-engaging component passes through the cardiac tissue separating the coronary sinus and the left atrium and moves through the left atrium substantially in parallel to a plane defined by a mitral valve annulus.

According to some embodiments of the invention, the engaging of the first mitral valve leaflet includes application of suction through the first leaflet-engaging component. According to some embodiments of the invention, the first leaflet-engaging component slides along a face of the first mitral valve leaflet. According to some embodiments of the invention, and further including the first leaflet-engaging component sliding along a face of an atrium wall prior to engaging of the first mitral valve leaflet.

According to some embodiments of the invention, further including engaging a second mitral valve leaflet. According to some embodiments of the invention, further including attaching an edge of the first mitral valve leaflet to an edge of the second mitral valve leaflet. According to some embodiments of the invention, the attaching is performed via the elongated catheter body.

According to some embodiments of the invention, the engaging of the second mitral valve leaflet is with a second leaflet-engaging component located proximal to the distal end of the leaflet-engaging device. According to some embodiments of the invention, the second leaflet-engaging component engages the second mitral valve leaflet at an atrial face of the second mitral valve leaflet. According to some embodiments of the invention, the engaging of the second mitral valve leaflet includes application of suction through the second leaflet-engaging component of the leaflet-engaging device. According to some embodiments of the invention, and further including the second leaflet-engaging component sliding along a face of the second mitral valve leaflet.

According to some embodiments of the invention, the first leaflet-engaging component and the second leaflet-engaging component of the leaflet-engaging device are independently maneuverable, and further including moving the first leaflet-engaging component and the second leaflet-engaging component one relative to the other so as to change the relative orientation of the engaged first leaflet and the engaged second leaflet.

According to some embodiments of the invention, further including directing a second leaflet-engaging device to proximity of the first mitral valve leaflet, and engaging the first mitral valve leaflet with the second leaflet-engaging device. According to some embodiments of the invention, further including directing a second leaflet-engaging device to proximity of a second mitral valve leaflet, and engaging a second mitral valve leaflet with the second leaflet-engaging device. According to some embodiments of the invention, the second leaflet-engaging device approaches the second leaflet substantially perpendicularly to a plane defined by an annulus of the mitral valve.

According to some embodiments of the invention, the first mitral valve leaflet is an anterior mitral valve leaflet and the second mitral valve leaflet is a posterior mitral valve leaflet. According to some embodiments of the invention, the first mitral valve leaflet is a posterior mitral valve leaflet and the second mitral valve leaflet is an anterior mitral valve leaflet.

According to some embodiments of the invention, the directing of the second leaflet-engaging device to proximity of the second mitral valve leaflet is through a left ventricle of the heart. According to some embodiments of the invention, the directing of the second leaflet-engaging device to proximity of the second mitral valve leaflet is through a left atrium of the heart.

According to some embodiments of the invention, further including, during (b), and/or (c) optically observing the atrial face of the mitral valve.

According to some embodiments of the invention, further including suturing a first mitral valve leaflet to a second mitral valve leaflet. According to some embodiments of the invention, further including clamping a first mitral valve leaflet to a second mitral valve leaflet. According to some embodiments of the invention, further including surgically operating on at least one mitral valve leaflet.

According to an aspect of some embodiments of the present invention there is provided a device for engaging cardiac valve leaflets, including a) an elongated catheter body with a proximal end and a distal end, the distal end shaped for passage into a heart of a mammalian body, and b) a first leaflet-engaging component located proximate to the distal end of the catheter body.

According to some embodiments of the invention, the first leaflet-engaging component includes a suction device.

According to some embodiments of the invention, the distal end is at an angle of between about 70° to 110° to a longitudinal axis of the elongated catheter body, the angle located proximally to the distal end. According to some embodiments of the invention, the angle is located at about 5 centimeters +/−20% from the distal end.

According to some embodiments of the invention, the elongated catheter body is configured to follow a catheter-guiding guide wire including a guide wire lumen passing from the proximal end to the distal end of the elongated catheter body, the guide wire lumen is configured for accepting the catheter-guiding guide wire, and wherein the guide wire lumen terminates at a guide wire port located substantially at a distal tip of the elongated catheter body.

According to some embodiments of the invention, the elongated catheter body includes a main lumen passing from a proximal end of the elongated catheter body and terminating at a port proximal to and at a side of the distal end of the elongated catheter body, the first-leaflet-engaging component is configured to move into and out of the main lumen through the port, and the first leaflet-engaging component is functionally associated with a first leaflet-engaging component director, the first leaflet-engaging component director passing from the proximal end to the distal end of the elongated catheter body and through the main lumen of the elongated catheter body, the first leaflet-engaging component director is configured so that manipulation of the first leaflet-engaging component director affects the moving of the first leaflet-engaging component into and out of the main lumen through the port.

According to some embodiments of the invention, the first leaflet-engaging component includes a suction port configured for communication with a vacuum source through a vacuum lumen passing through the elongated catheter body.

According to some embodiments of the invention, further including a second leaflet-engaging component located proximate to the distal end of the catheter body. According to some embodiments of the invention, the second leaflet-engaging component is configured to move into and out of the main lumen through the port.

According to some embodiments of the invention, the second leaflet-engaging component is functionally associated with a second leaflet-engaging component director, the second leaflet-engaging component director passing from the proximal end to the distal end of the elongated catheter body and through the main lumen of the elongated catheter body, the second leaflet-engaging component director configured so that manipulation of the second leaflet-engaging component director affects the moving of the second leaflet-engaging component into and out of the main lumen through the port.

According to some embodiments of the invention, the second leaflet-engaging component includes a suction port configured for communication with a vacuum source through a vacuum lumen passing through the elongated catheter body.

According to some embodiments of the invention, further including a reversible anchoring component proximate to the distal end of the elongated catheter body, the anchoring component having at least two states i) a first non-anchoring state, and ii) a second anchoring state, wherein the anchoring component engages walls of a coronary sinus in which the distal end is located so as stabilize a position of the distal end in the coronary sinus, and an anchoring component actuator functionally associated with the anchoring component, configured to allow changing of the anchoring component from the first non-anchoring state to the second anchoring state.

According to some embodiments of the invention, the reversible anchoring component includes an inflatable balloon. According to some embodiments of the invention, the inflatable balloon envelops the elongated catheter body, and includes a side opening allowing a component to move into and out of the main lumen through the port and through the side opening. According to some embodiments of the invention, the anchoring component is configured to avoid complete obstruction of a lumen of the coronary sinus when in the second anchoring state.

According to some embodiments of the invention, further including a suturing component for suturing a first mitral valve leaflet to a second mitral valve leaflet. According to some embodiments of the invention, further including a clamping component for clamping a first mitral valve leaflet to a second mitral valve leaflet.

According to some embodiments of the invention, further including a leaflet augmentation component for augmenting at least one mitral valve leaflet.

According to some embodiments of the invention, further including a cardiac valve obstructor for obstructing at least part of a cardiac valve.

According to an aspect of some embodiments of the present invention there is provided a kit including the device for engaging cardiac valve leaflets, and a separate, additional suturing device for suturing a first mitral valve leaflet to a second mitral valve leaflet.

According to some embodiments of the invention, a kit including the device for engaging cardiac valve leaflets, and a separate, additional clamping device for clamping a first mitral valve leaflet to a second mitral valve leaflet.

According to some embodiments of the invention, a kit including the device for engaging cardiac valve leaflets, and a separate, additional leaflet augmentation device for augmenting at least one mitral valve leaflet.

According to some embodiments of the invention, a kit including the device for engaging cardiac valve leaflets, and a separate, additional cardiac valve obstructor for obstructing at least part of a cardiac valve.

According to an aspect of some embodiments of the present invention there is provided a device suitable for defining a minimally invasive conduit to a left atrium of a mammalian heart, including a) an elongated catheter body with a proximal end, a distal end and a distal tip, the distal end configured for passage into a coronary sinus from a peripheral region of a mammalian body, b) a main lumen passing from the proximal end to the distal end of the elongated catheter body, the main lumen terminating at a side port located at a side of the distal end of the elongated catheter body, c) a reversible anchoring component in proximity of the distal end of the elongated catheter body, the anchoring component having at least two states i) a first non-anchoring state, and ii) a second anchoring state, wherein the anchoring component engages walls of a coronary sinus in which the distal end is located so as stabilize a position of the distal end in the coronary sinus, and d) an anchoring component actuator functionally associated with the anchoring component, configured to allow changing of the anchoring component from the first non-anchoring state to the second anchoring state, wherein the anchoring component is configured to anchor on a side of the elongated catheter body opposite the side port.

According to some embodiments of the invention, further including an elongated piercing component configured for passage through the main lumen from the proximal end to emerge out through the side port so as to puncture tissue proximate to the side port.

According to some embodiments of the invention, the elongated piercing component is a component discrete from the elongated catheter body. According to some embodiments of the invention, the elongated catheter body is configured for passage of the distal end into a coronary sinus so that the side port faces a cardiac wall separating the coronary sinus from a left atrium.

According to some embodiments of the invention, the distal end is at an angle of between about 70° to 110° to a longitudinal axis of the elongated catheter body, the angle located proximally to the distal end. According to some embodiments of the invention, the angle is located at about 5 centimeters +/−20% from the distal end.

According to an aspect of some embodiments of the present invention there is provided a method for defining a minimally invasive conduit to a left atrium of a mammalian heart, including a) providing a device including i) an elongated catheter body with a proximal end, a distal end and a distal tip, the distal end configured for passage into a coronary sinus from a peripheral region of a mammalian body, and ii) a main lumen passing from the proximal end to the distal end of the elongated catheter body, the main lumen terminating at a side port located at a side of the distal end of the elongated catheter body; b) directing the distal end of the elongated catheter body of the device into a coronary sinus of a heart so that the side port faces a cardiac wall separating the coronary sinus from a left atrium, and c) passing an elongated piercing component through the side port so as to puncture the cardiac wall separating the coronary sinus from a left atrium.

According to some embodiments of the invention, the heart is a beating heart.

According to some embodiments of the invention, the distal end of the elongated body of the leaflet-engaging device is directed into the coronary sinus through a wall of the coronary sinus. According to some embodiments of the invention, further including engaging the walls of the coronary sinus so as stabilize a position of the distal end in the coronary sinus. According to some embodiments of the invention, further including pressing the side port against the cardiac wall separating the coronary sinus from the left atrium.

According to some embodiments of the invention, further including, passing an object through at least part of the main lumen to the side port. According to some embodiments of the invention, the passing is into the left atrium through the side port. According to some embodiments of the invention, the object includes an object selected from the group consisting of a medical device, an observation device and a sensor. According to some embodiments of the invention, the object includes an object selected from the group consisting of a fluid, a composition and an active agent.

According to an aspect of some embodiments of the present invention there is provided a method for reducing the mobility of a mitral valve leaflet, comprising: a) directing a distal end of an elongated catheter body of a leaflet-engaging device into a coronary sinus of a heart; b) passing a first leaflet-engaging component located proximate to the distal end of the elongated catheter body of the leaflet-engaging device through cardiac tissue separating the coronary sinus and a left atrium of the heart to enter a left atrium of the heart (e.g., through a coronary sinus puncture); and c) engaging a first mitral valve leaflet with the first leaflet-engaging component thereby reducing the mobility of the at least one mitral valve leaflet.

In some embodiments, the heart is a human heart. In some embodiments, the heart is of a non-human animal. In some embodiments, the heart is a beating heart. In some embodiments, the heart is not beating. In some embodiments, the heart is of a non-living animal.

In some embodiments, the distal end of the elongated catheter body of the leaflet-engaging device is directed through a superior vena cava, into a right atrium to enter the coronary sinus through a respective cardiac ostium.

In some embodiments, the distal end of the elongated body of the leaflet-engaging device is directed through an inferior vena cava, into a right atrium to enter the coronary sinus through a respective cardiac ostium.

In some embodiments, the distal end of the elongated body of the leaflet-engaging device is directed into the coronary sinus through an external wall of the coronary sinus.

In some embodiments, the first leaflet-engaging component engages the first mitral valve leaflet from an atrial face of the first mitral valve leaflet.

In some embodiments, prior to the engaging of the first mitral valve leaflet, the first leaflet-engaging component passes through the cardiac tissue separating the coronary sinus and the left atrium and moves through the left atrium substantially in parallel to a plane defined by a mitral valve annulus.

In some embodiments, the engaging is of an atrial face (as opposed to an edge) of the first mitral valve leaflet.

In some embodiments, engaging the first mitral valve leaflet comprises application of suction through the first leaflet-engaging component.

In some embodiments, the method further comprises engaging a second mitral valve leaflet. In some embodiments, the method further comprises securing an edge of the first mitral valve leaflet to an edge of the second mitral valve leaflet, in some embodiments thereby performing a “bow-tie” repair of a mitral valve.

In some embodiments, engaging of the second mitral valve leaflet is with a second leaflet-engaging component located proximal to the distal end of the leaflet-engaging device.

In some embodiments, a second leaflet-engaging component engages the second mitral valve leaflet from an atrial face of the second mitral valve leaflet

In some embodiments, prior to engaging of the second mitral valve leaflet, the second leaflet-engaging component passes through the cardiac tissue separating the coronary sinus and the left atrium and moves through the left atrium substantially in parallel to a plane defined by a mitral valve annulus.

In some embodiments, the engaging of the second mitral valve leaflet is of an atrial face (as opposed to an edge) of the second mitral valve leaflet.

In some embodiments, the engaging of the second mitral valve leaflet comprises application of suction through the second leaflet-engaging component of the leaflet-engaging device.

In some embodiments, the first leaflet-engaging component and the second leaflet-engaging component of the leaflet-engaging device are independently maneuverable, and the method further comprises moving the first leaflet-engaging component and the second leaflet-engaging component one relative to the other so as to change the relative orientation of the engaged first leaflet and the engaged second leaflet.

In some embodiments, the method further comprises directing a second leaflet-engaging device to proximity of a second mitral valve leaflet and engaging a second mitral valve leaflet with the second leaflet-engaging device.

In some embodiments, the second leaflet-engaging device approaches the second leaflet substantially perpendicularly to a plane defined by an annulus of the mitral valve.

In some embodiments, the first mitral valve leaflet is an anterior mitral valve leaflet and the second mitral valve leaflet is a posterior mitral valve leaflet. In some embodiments, the first mitral valve leaflet is a posterior mitral valve leaflet and the second mitral valve leaflet is an anterior mitral valve leaflet.

In some embodiments, directing of the second leaflet-engaging device to proximity of the second mitral valve leaflet is through a left ventricle of the heart. In some embodiments, the second leaflet-engaging device enters the left ventricle by passing through an aorta of the heart into the left ventricle (e.g., a percutaneous retrograde approach, entering through a subclavian, carotid or femoral artery). In some embodiments, the second leaflet-engaging device enters the left ventricle by passing through an apex of the heart into the left ventricle.

In some embodiments, directing of the second leaflet-engaging device to proximity of the second mitral valve leaflet is through a left atrium of the heart. In some embodiments, the second leaflet-engaging device enters the left atrium by passing through an intraatrial septum from a right atrium of the heart into the left atrium, e.g. a percutaneous antegrade approach with a transseptal puncture, generally through the fossa ovalis. In some embodiments, the second leaflet-engaging device enters the left atrium by passing through a roof of the left atrium.

In some embodiments, the method further comprises, during (b) the passing of the first leaflet-engaging component through cardiac tissue to enter a left atrium of the heart and/or (c) the engaging of the first mitral valve leaflet with the first leaflet-engaging component, optically observing the atrial face of the mitral valve.

According to an aspect of some embodiments of the present invention there is also provided a method for defining a minimally invasive conduit to a left atrium of a mammalian heart, comprising: a) providing a device including: i) an elongated catheter body with a proximal end, a distal end and a distal tip, the distal end configured for passage into a coronary sinus from a peripheral region of a mammalian body; and ii) a main lumen passing from the proximal end to the distal end of the elongated catheter body, the main lumen terminating at a side port located at a side of the distal end of the elongated catheter body; b) directing the distal end of the elongated catheter body of the device into a coronary sinus of a heart so that the side port faces a cardiac wall separating the coronary sinus from a left atrium; and c) passing a piercing component through the side port so as to puncture the cardiac wall separating the coronary sinus from a left atrium.

In some embodiments, the heart is a human heart. In some embodiments, the heart is of a non-human animal. In some embodiments, the heart is a beating heart. In some embodiments, the heart is not beating. In some embodiments, the heart is of a non-living animal.

In some embodiments, the distal end of the elongated body of the leaflet-engaging device is directed through a superior vena cava, into a right atrium to enter the coronary sinus through a respective cardiac ostium.

In some embodiments, the distal end of the elongated body of the leaflet-engaging device is directed through an inferior vena cava, into a right atrium to enter the coronary sinus through a respective cardiac ostium.

In some embodiments, the distal end of the elongated body of the leaflet-engaging device is directed into the coronary sinus through a wall of the coronary sinus.

In some embodiments, the method further comprises: engaging the walls of the coronary sinus so as stabilize a position of the distal end of the elongated catheter body in the coronary sinus.

In some embodiments, the method further comprises: pressing (in embodiments, sealingly pressing) the side port against the cardiac wall separating the coronary sinus from the left atrium.

In some embodiments, the method further comprises passing an object through at least part of the main lumen to the side port of the elongated catheter body. In some embodiments, the passing is from the proximal end of the elongated catheter body to the side port. In some embodiments, the passing is into the left atrium through the side port. In some embodiments, the object passed comprises an object such as a medical device, an observation device or a sensor. In some embodiments, the object passed is an object such as a fluid, a composition or an active agent. In some such embodiments such passing is effective administration of the object to the patient.

According to an aspect of some embodiments of the present invention there is also provided a device for reducing the mobility of a mitral valve leaflet, comprising: a) an elongated catheter body with a proximal end and a distal end, the distal end configured for passage into a coronary sinus from a peripheral region of a mammalian body; and b) a first leaflet-engaging component located proximate to the distal end of the catheter body.

In some embodiments, the diameter of the elongated catheter body at the distal end is no more than about 7 mm (21 French), no more than about 6 mm and even no more than about 5 mm.

In some embodiments, the elongated catheter body is configured to follow a catheter-guiding guide wire. In some embodiments, the configuration to follow a catheter-guiding guide wire comprises a guide wire lumen passing from the proximal end to the distal end of the elongated catheter body, the lumen configured for accepting a catheter-guiding guide wire. In some embodiments, the catheter-guiding guide wire lumen is a dedicated lumen. In some embodiments, the lumen has an additional function, see below. In some embodiments, the catheter-guiding guide wire lumen terminates at a guide wire port located substantially at a distal tip of the elongated catheter body.

In some embodiments, the elongated catheter body comprises a main lumen passing from a proximal end of the elongated catheter body and terminating at a port proximal to the distal end of the elongated catheter body; wherein the first-leaflet-engaging component is configured to move into and out of the main lumen through the port. In some embodiments, the port is located at a side of the distal end of the elongated catheter body.

In some embodiments, the first leaflet-engaging component is functionally associated with a first leaflet-engaging component director, the first leaflet-engaging component director passing from the proximal end to the distal end of the elongated catheter body and through the main lumen of the elongated catheter body, the first leaflet-engaging component director configured so that manipulation of the first leaflet-engaging component director affects the moving of the first leaflet-engaging component into and out of the main lumen through the port.

In some embodiments, the first leaflet-engaging component comprises a suction port configured for communication with a vacuum source through a first vacuum lumen passing through the first leaflet-engaging component director. In some embodiments, the first leaflet-engaging component comprises a suction port configured for communication with a vacuum source through a vacuum lumen passing through the elongated catheter body.

In some embodiments, the device further comprises a second leaflet-engaging component located proximate to the distal end of the catheter body.

In some embodiments, the elongated catheter body comprises a main lumen passing from a proximal end of the catheter body and terminating at a port proximal to the distal end of the elongated catheter body; and wherein the second leaflet-engaging component is configured to move into and out of the main lumen through the port. In some embodiments, the port is located at a side of the distal end of the elongated catheter body.

In some embodiments, the second leaflet-engaging component is functionally associated with a second leaflet-engaging component director, the second leaflet-engaging component director passing from the proximal end to the distal end of the elongated catheter body and through the main lumen of the elongated catheter body, the second leaflet-engaging component director configured so that manipulation of the second leaflet-engaging component director affects the moving of the second leaflet-engaging component into and out of the main lumen through the port.

In some embodiments, the second leaflet-engaging component comprises a suction port configured for communication with a vacuum source through a second vacuum lumen passing through the second leaflet-engaging component director. In some embodiments, the second leaflet-engaging component comprises a suction port configured for communication with a vacuum source through a vacuum lumen passing through the elongated catheter body.

In some embodiments where one or more leaflet-engaging components of a device of the present invention comprise a vacuum port to engage a leaflet through suction, the device is generally provided with an actuator functionally associated with the leaflet-engaging components to allow the suction to be activated, deactivated and in some embodiments varied in intensity. In some embodiments, each vacuum port may be activated separately. In some embodiments, two or more vacuum ports are configured to be activated simultaneously.

In some embodiments, the device further comprises: a reversible anchoring component proximate to the distal end of the elongated catheter body, the anchoring component having at least two states: i) a first non-anchoring state; and ii) a second anchoring state, wherein the anchoring component engages walls of a coronary sinus in which the distal end is located so as stabilize a position of the distal end in the coronary sinus; and an anchoring component actuator functionally associated with the anchoring component, configured to allow changing of the anchoring component from the first non-anchoring state to the second anchoring state, generally when the device is inside a coronary sinus. In some embodiments, the activation of the activator is from the proximal end of the catheter body.

In some embodiments, the anchoring component is an inflatable balloon wherein the second anchoring state is an expanded state of the balloon and the first non-anchoring state is a less or non-expanded state of the balloon. In some embodiments, wherein the anchoring component actuator comprises at least one inflation lumen in fluid communication with the inflatable balloon, configured to transport an inflation fluid to and from the balloon so as to change the state of the balloon.

In some embodiments, the anchoring component is configured to avoid complete obstruction of a lumen of a coronary sinus when in the second anchoring state. In some embodiments, the anchoring component is an inflatable balloon wherein the second anchoring state is an expanded state of the balloon where the balloon optionally has an acircular cross-section and the first non-anchoring state is a less or non-expanded state of the balloon. In some embodiments, the inflatable balloon has a side port, corresponding to a side port in the elongated catheter body. Having the anchor located at the same position as the side port which is used for punching through the wall of the left ventricle provides anchoring which does not move relative to the punching location, even while the heart is beating.

In some embodiments, a device includes a side port from which a leaflet-engaging component moves in and out, and also includes an anchoring component, the anchoring component situated on a side of the elongated catheter body opposite the side port and is configured not to obstruct the side port when in an anchoring state. In some embodiments, such a configuration leads to the anchoring component pressing the side port against the wall of a coronary sinus in which deployed.

In some embodiments, the elongated catheter body is configured for passage of the distal end into a coronary sinus so that the first leaflet-engaging component is in proximity of a mitral valve by entering a mammalian body through an incision in the mammalian body into a right atrium to enter the coronary sinus through a cardiac ostium.

In some embodiments, the elongated body is configured for passage of the distal end of the catheter body into a coronary sinus by entering a femoral vein through the incision, through an inferior vena cava, into a right atrium to enter the coronary sinus through a cardiac ostium.

In some embodiments, the elongated body is configured for passage of the distal end of the catheter body into a coronary sinus by entering a jugular vein through the incision, through a superior vena cava, into a right atrium to enter the coronary sinus through a cardiac ostium.

In some embodiments, the elongated body is configured for passage of the distal end of the catheter body into a coronary sinus by entering a subclavian vein through the incision, through a superior vena cava, into a right atrium to enter the coronary sinus through a cardiac ostium.

In some embodiments, the elongated body is configured for passage of the distal end of the catheter body into a coronary sinus by entering a mammalian body through an incision in the thorax of the mammalian body to enter the coronary sinus through a wall of the coronary sinus.

According to the teachings of the present invention there is also provided a device suitable for defining a minimally invasive conduit to a left atrium of a mammalian heart, comprising: a) an elongated catheter body with a proximal end, a distal end and a distal tip, the distal end configured for passage into a coronary sinus from a peripheral region of a mammalian body; and d) a main lumen passing from the proximal end to the distal end of the elongated catheter body, the main lumen terminating at a side port located at a side of the distal end of the elongated catheter body.

In some embodiments, the diameter of the elongated catheter body at the distal end is no more than about 7 mm (21 French), no more than about 6 mm (18 French) and even no more than about 5 mm (15 French).

In some embodiments, the device further comprises: c) a reversible anchoring component in proximity of the distal end of the elongated catheter body, the anchoring component having at least two states: i) a first non-anchoring state; and ii) a second anchoring state, wherein the anchoring component engages walls of a coronary sinus in which the distal end is located so as stabilize a position of the distal end in the coronary sinus; and d) an anchoring component actuator functionally associated with the anchoring component, configured to allow changing of the anchoring component from the first non-anchoring state to the second anchoring state.

In some embodiments, the anchoring component is an inflatable balloon and the second anchoring state is an expanded state of the balloon and the first non-anchoring state is a less or non-expanded state of the balloon

In some embodiments, the inflatable balloon is situated on a side of the elongated catheter body opposite the side port and is configured to not obstruct the side port when in an anchoring state.

In some embodiments, the anchoring component actuator comprises at least one inflation lumen in fluid communication with the inflatable balloon, configured to transport an inflation fluid to and from the balloon so as to change the state of the balloon.

In some embodiments, the anchoring component is configured to avoid complete obstruction of the lumen of a coronary sinus when in the second anchoring state.

In some embodiments, the anchoring component is an inflatable balloon wherein the second anchoring state is an expanded state of the balloon where the balloon optionally has an acircular cross-section and the first non-anchoring state is a less or non-expanded state of the balloon

In some embodiments, the elongated body is configured to follow a catheter-guiding guide wire. In some embodiments, the configuration to follow a catheter-guiding guide wire comprises a guide wire lumen passing from the proximal end to the distal end of the elongated catheter body, the guide wire lumen configured for accepting a catheter-guiding guide wire. In some embodiments, the catheter-guiding guide wire lumen and the main lumen are substantially the same lumen. In some embodiments, the catheter-guiding guide wire lumen and the main lumen are different lumens. In some embodiments, the catheter-guiding guide wire lumen terminates at a guide wire port located substantially at a distal tip of the elongated catheter body.

In some embodiments, the device further comprises an elongated piercing component configured for passage through the main lumen from the proximal end to emerge out through the side port so as to puncture tissue proximate to the side port. In embodiments, the elongated piercing component is a component discrete from the elongated catheter body.

In some embodiments, the elongated catheter body is configured for passage of the distal end of the catheter body into a coronary sinus so that the side port faces a cardiac wall separating the coronary sinus from a left atrium by entering a mammalian body through an incision in the mammalian body into a right atrium to enter the coronary sinus through a cardiac ostium.

In some embodiments, the elongated body is configured for passage of the distal end of the catheter body into a coronary sinus by entering a femoral vein through the incision, through an inferior vena cava, into a right atrium to enter the coronary sinus through a cardiac ostium.

In some embodiments, the elongated body is configured for passage of the distal end of the catheter body into a coronary sinus by entering a jugular vein through the incision, through a superior vena cava, into a right atrium to enter the coronary sinus through a cardiac ostium.

In some embodiments, the elongated body is configured for passage of the distal end of the catheter body into a coronary sinus by entering a subclavian vein through the incision, through a superior vena cava, into a right atrium to enter the coronary sinus through a cardiac ostium.

In some embodiments, the elongated body is configured for passage of the distal end of the catheter body into a coronary sinus by entering a mammalian body through an incision in the thorax of the mammalian body to enter the coronary sinus through a wall of the coronary sinus.

Unless otherwise defined, all technical and scientific terms used herein have the same meaning as commonly understood by one of ordinary skill in the art to which this invention pertains. In case of conflict, the patent specification, including definitions, will control.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

As used herein, the indefinite articles “a” and “an” mean “at least one” or “one or more” unless the context clearly dictates otherwise.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

### BRIEF DESCRIPTION OF THE FIGURES

Some embodiments of the invention are herein described, by way of example only, with reference to the accompanying figures. The description, together with the figures, makes apparent how embodiments of the invention may be practiced to those skilled in the art. It is stressed that the particulars shown in the figures are by way of example and for purposes of illustrative discussion of embodiments of the invention.

In the figures:

FIG. 1 (prior art) is a schematic depiction of a healthy heart in cross-section;

FIGS. 2A and 2B (prior art) depict a mitral valve of a healthy heart;

FIGS. 3A and 3B (prior art) depict a mitral valve of a heart suffering from ischemic mitral regurgitation related to incomplete coaptation of the leaflets of the mitral valve;

FIG. 4A (prior art) depicts a mitral valve of a heart having undergone a “bow tie” repair according to Alfieri et al.;

FIG. 4B (prior art) is a schematic depiction of a transcatheter mitral valve therapy device inserted into the left ventricle through the left atrium;

FIG. 4C is a schematic depiction of approaches to a left atrium;

FIGS. 5A-5H are schematic depictions of a first embodiment of the present invention relating to a minimally invasive conduit to the left atrium;

FIGS. 6A-6J are schematic depictions of a second embodiment of the present invention relating to reducing the mobility of mitral valve leaflets;

FIG. 6K is a schematic depiction of the embodiment of FIGS. 6A-6J, after the mitral valve leaflets have been engaged by the leaflet-engaging components;

FIGS. 7A, 7B, and 7C are schematic depictions of part of an alternative embodiment of FIGS. 6A-6K, at various extensions from an elongated catheter body;

FIG. 8 is a schematic depiction of the embodiment of FIGS. 6A-6K and 7, in use for reducing the mobility of mitral valve leaflets while a mitral valve procedure is being performed;

FIG. 9 is a schematic depiction of a third embodiment of the present invention relating to mitral valve leaflet augmentation;

FIG. 10A is a schematic depiction of a fourth embodiment of the present invention relating to a mitral valve obstruction device; and

FIG. 10B is a schematic depiction of the fourth embodiment of FIG. 10A, deployed in a heart.

### DESCRIPTION OF EMBODIMENTS

The present invention, in some embodiments thereof, relates to the fields of cardiac surgery and cardiology. Some embodiments of the present invention relate to access of the left atrium or the mitral valve of a mammalian heart through the coronary sinus. Some embodiments of the present invention relate to reduction of the mobility of one or both mitral valve leaflets.

In some embodiments, the teachings of some embodiments of the present invention allow for reduction of mobility of one or both mitral valve leaflets, for example to the extent that performance of surgical intervention of the mitral valve, for example deployment of a leaflet-edge capturing device to perform a “bow-tie” repair of the mitral valve is relatively quick, simple and accurate. Specifically, some embodiments of the present invention relate to methods and devices to capture and reduce the mobility of one or more mitral valve leaflets. Some embodiments of the present invention are useful in reducing the mobility of one or more mitral valve leaflets that in some embodiments is useful for treating a condition where such immobilization is beneficial. Some embodiments of the teachings of the present invention are useful in treating conditions relating to malfunctioning mitral valves, for example mitral valves having insufficient leaflet coaptation. In some such embodiments, one or both leaflets are captured and the mobility thereof reduced to the extent that performing a surgical intervention on the leaflet, for instance performing edge-to-edge securing of the leaflets, is simplified. Another instance includes inserting a mitral valve obstruction device such as described in the related U.S. Provisional Patent Application No. 61/041,942 mentioned above.

By treating a condition is included curing and preventing the condition; treating, curing, preventing appearance of or ameliorating effects and/or symptoms of the condition, whether of clinical or esthetic significance; and inhibiting, slowing or reversing progression of the condition, effects of the condition and/or symptoms of the condition, whether of clinical or esthetic significance.

In some embodiments, the teachings of some embodiments of the present invention provide for a minimally invasive conduit to the left atrium, for any number of purposes including the observation of the inside of the left atrium and/or the mitral valve, access to other portions of the heart or cardiovasculature, and others. In some embodiments, the provided conduit is useful for the administration of active agents, which in some embodiments is useful in treating conditions where administration of an active agent through the conduit is beneficial.

In some embodiments, an active agent is administered, for example to treat a condition. Herein, the term “active agent” is understood to include chemical, biological or pharmaceutical entities including any natural or synthetic chemical or biological substance. Typical active entities include but are not limited to active pharmaceutical ingredients, antibodies, antigens, biological materials, chemical materials, chromatogenic compounds, contrasting agents, drugs, enzymes, fluorescent probes, immunogenes, indicators, ligands, nucleic acids, nutrients, peptides, physiological media, proteins, selective toxins and toxins.

Exemplary embodiments of the invention are discussed hereinbelow with reference to specific materials, methods and examples. The material, methods and examples discussed herein are illustrative and not intended to be limiting. In some embodiments, methods and materials similar or equivalent to those described herein are used in the practice or testing of embodiments of the invention. It is to be understood that the invention is not necessarily limited in its application to the details of construction and the arrangement of the components and/or methods set forth in the following description and/or illustrated in the drawings. The invention is capable of other embodiments or of being practiced or carried out in various ways.

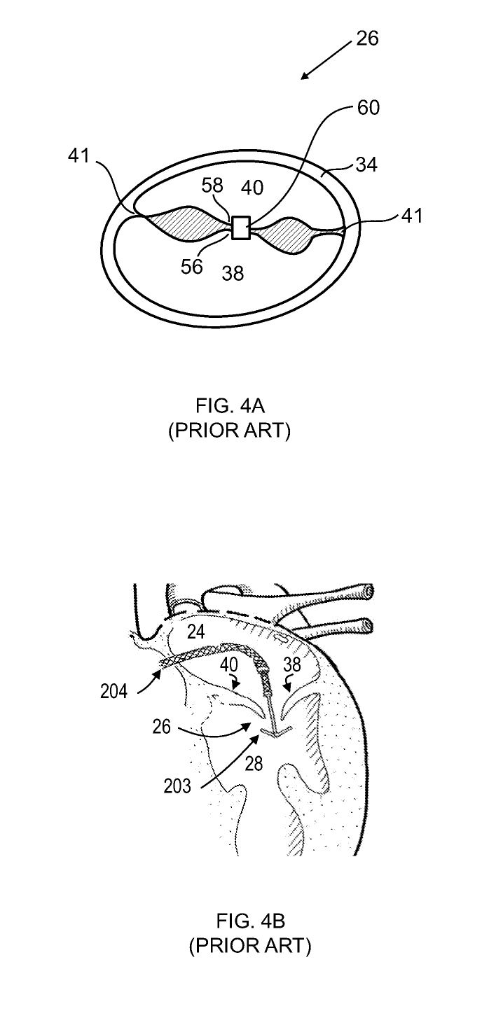

Reference is now additionally made to FIG. 4C, which is a schematic depiction of approaches to a left atrium 24.

One approach includes using a catheter 201 to punch through an interatrial septum 33 between the right atrium 12 and the left atrium 24, thereby entering the left atrium 24.

Another approach includes using a catheter 202 to reach the left atrium 24 in a retrograde fashion, through the aorta 32. The catheter 202 is pushed upstream through the aorta 32, through the aortic semi-lunar valve 30, then through the mitral valve 26 into the left atrium 24.

### Minimally-Invasive Conduit to the Left Atrium

In some embodiments, the teachings of some embodiments of the present invention provide methods and devices relating to providing a minimally invasive conduit to the left atrium, for any number of purposes.

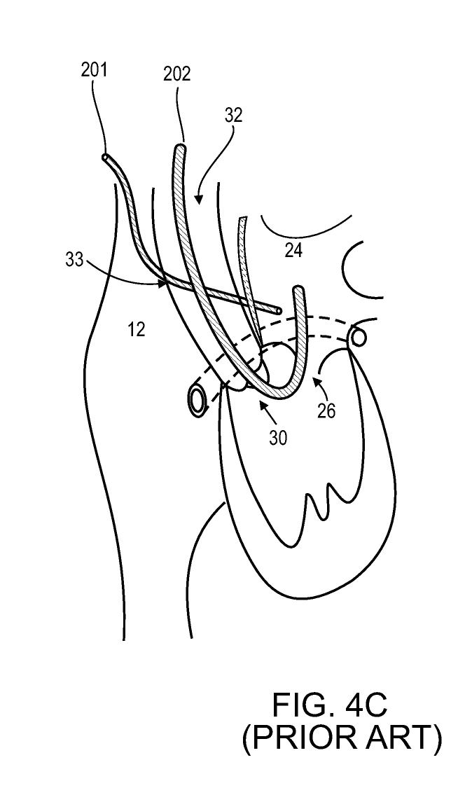

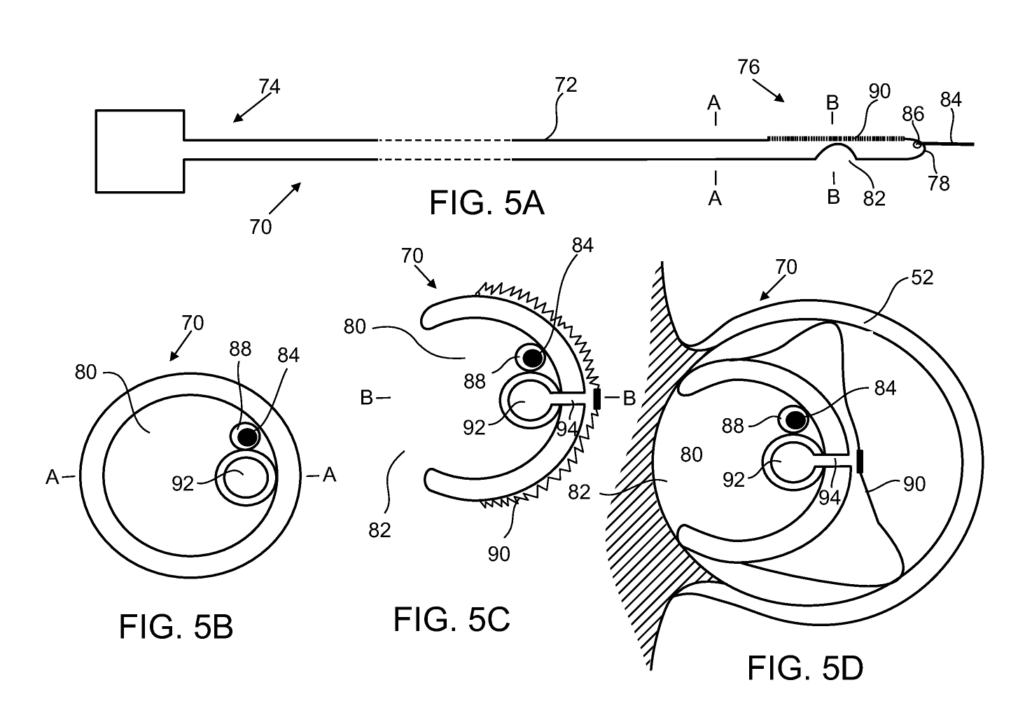

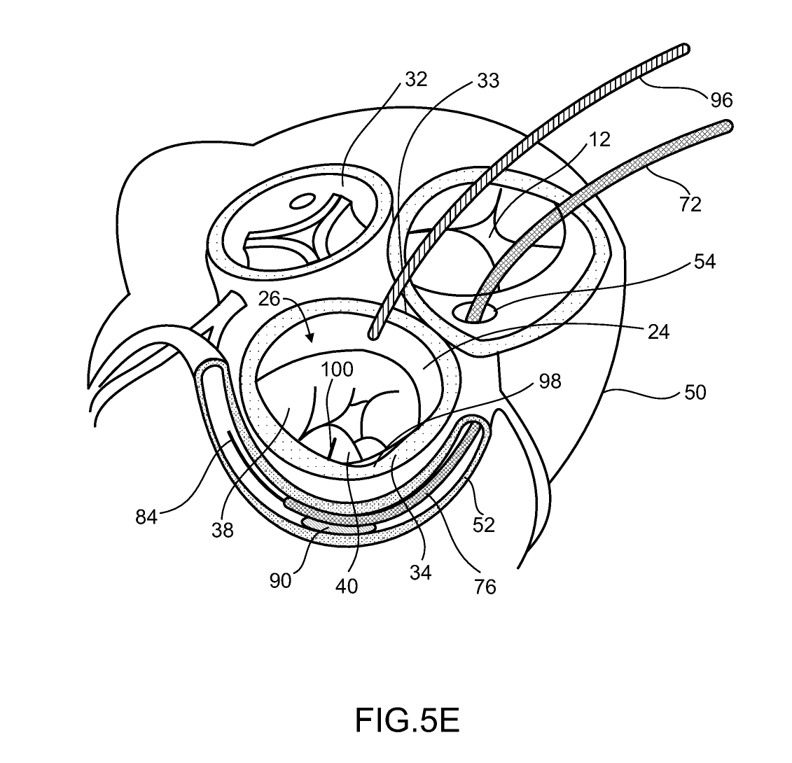

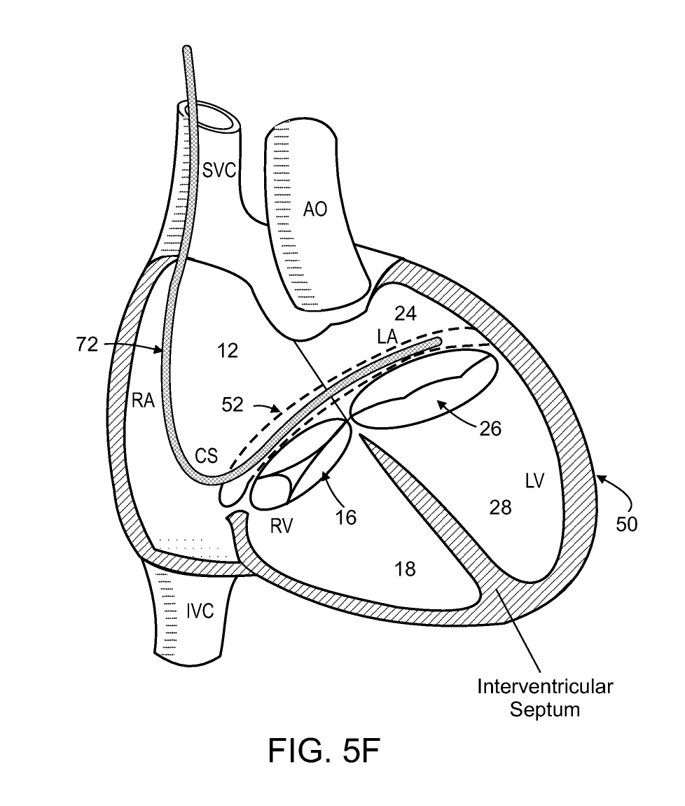

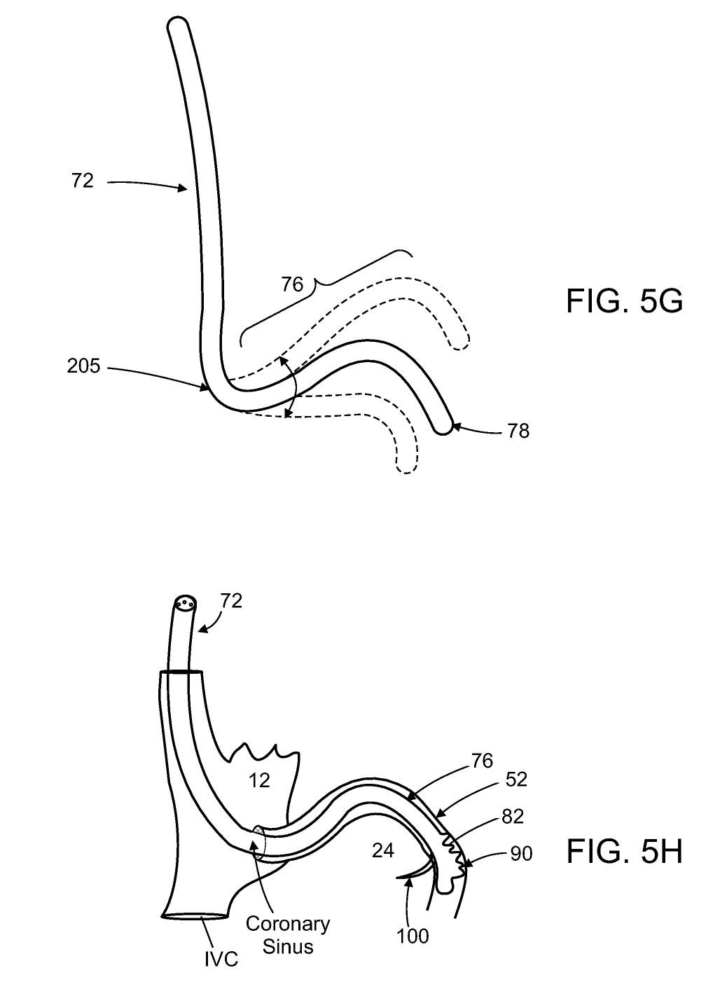

An embodiment of a device suitable for defining a minimally invasive conduit to a left atrium of a mammalian heart, device 70, is depicted in FIG. 5A (side view), in FIG. 5B (cross section A-A) and in FIG. 5C (cross section B-B). Device 70 is also depicted deployed in a coronary sinus 52 of heart 50 in FIG. 5D (cross section B-B) and FIG. 5E. In FIG. 5E, heart 50 is depicted from a superior view with most of the atrial walls removed to expose aorta 32, right atrium 12, left atrium 24, anterior leaflet 38, posterior leaflet 40 and mitral valve annulus 34 of mitral valve 26, a portion of interatrial septum 33, cardiac ostium 54 and coronary sinus 52 from which a portion is removed to show the lumen thereof. FIG. 5F depicts the heart 50 in a simplified cross-sectional side view, depicting the elongated catheter body 72 passing through the coronary sinus 52 relative to the right atrium 12, the left atrium 24, the right ventricle 18, and the left ventricle 28, and relative to the tricuspid valve 16 and the mitral valve 26.

Device 70 is substantially a cardiac catheter and comprises an elongated catheter body 72 with a proximal end 74, a distal end 76 and a distal tip 78.

Device 70 and particularly catheter body 72 is configured in terms of length, flexibility and maneuverability to enter the cardiovascular system from the right jugular vein, to pass through a superior vena cava, a right ventricle and to enter a coronary sinus through a cardiac ostium.

Catheter body 72 is substantially a 5 mm (15 French) catheter tube defining a main lumen 80. The 5 mm outer diameter of distal end 76 is narrow enough to easily enter most adult coronary sinuses, which typically have a diameter of between 7 and 10 mm. Main lumen 80 passes from proximal end 74 and terminates at side port 82, located at the side of catheter body 72 near distal end 76 of catheter body 72.

Catheter body 72 is configured for guidance with a catheter-guiding guide wire 84. Passing from proximal end 74 out through a guide wire port 86 at distal tip 78 of catheter body 72 through main lumen 80 is a catheter-guiding guide wire lumen 88 defined by a 0.54 mm (0.021″) tube. Catheter-guiding guide wire lumen 88 is configured to accept a 0.46 mm (0.016″, 1.2 French) catheter-guiding guide wire 84 (e.g., Fathom®-16 guide wire, Boston Scientific Corporation, Boston, Mass., USA).

On the side of catheter body 72 and opposite side port 82 is an inflatable balloon 90 configured to function as a reversible anchoring component. When balloon 90 is in a first state (not or only slightly inflated) balloon 90 has a low profile allowing passage through the vascular system. When balloon 90 is in a second state (inflated), the walls of balloon 90 expand outwards and adopt a higher profile. When balloon 90 is found in a coronary sinus 52 in the second inflated state (FIGS. 5D and 5E), balloon 90 engages the luminal walls of coronary sinus 52, stabilizing the position of distal end 76 and side port 82 inside coronary sinus 52. Balloon 90 is expandable to a maximal diameter of about 11 mm (33 French), sufficient for engaging even the largest typical coronary sinus having a diameter of about 10 mm. In some embodiments, a larger diameter expandable balloon is provided as an anchoring component.

In some embodiments, the balloon 90 has a side port, corresponding to the side port 82 in the catheter body 72. Having the anchor located at the same position as the side port 82 which is used for punching through the wall of the left ventricle provides anchoring which does not move relative to the punching location, even while the heart is beating.

When balloon 90 is found in a coronary sinus 52 in the second inflated state (FIGS. 5D and 5E), the asymmetric disposition of balloon 90 on the side of catheter body 72 opposite side port 82 not only stabilizes the position of side port 82 in coronary sinus 52 but also sealingly presses side port 82 against the luminal wall of coronary sinus opposite left atrium 24.

As seen in FIG. 5D, inflatable balloon 90 optionally has an acircular cross section in the second inflated state, specifically having a bidentate or stellate cross section. Thus when balloon 90 is in a second anchoring state inside a coronary sinus 52, balloon 90 avoids complete obstruction of the lumen of coronary sinus 52.

Balloon 90 is in fluid communication with an inflation lumen 92 passing from proximal end 74 to distal end 76 through inflation port 94. Control of the degree of inflation of balloon 90 and thus whether balloon 90 is in a first non-anchoring state or in a second anchoring state is provided by an actuator (not depicted) functionally associated with a source (not depicted) of inflation fluid (e.g., saline) which forces inflation fluid into or removes inflation fluid out of balloon 90 through inflation lumen 92 in the manner of inflatable balloons known in the art of angioplasty. Both the actuator and the source of inflation fluid are associated with inflation lumen 92 at proximal end 74.

As with some cardiac catheters known in the art, at proximal end 74 may be located components such as interfaces, connectors and ports necessary operation of device 70. Such components include, by way of a non-limiting example, a proximal port of catheter-guiding guide wire lumen 88, of main lumen 80, and of inflation lumen 92.

An embodiment of a method for defining a minimally invasive conduit to a left atrium of a mammalian heart is discussed in detail with reference to device 70, FIGS. 5A, 5B, 5C and especially FIGS. 5D 5E, and 5F.

A human patient with heart 50 is prepared for definition of a minimally invasive path to the left atrium of heart 50, including, by way of a non-limiting example, by optional deployment of a trans esophageal echocardiograph (TEE) probe, and/or an optical observation device comprising an optical fiber bundle 96 (e.g., a 1 mm outer diameter leached fiber optic image bundle available from Schott AG, Mainz, Germany) entering the body of the patient from the right femoral vein, through the inferior vena cava, into right atrium 12 and penetrating into left atrium 24 via a cannula (not depicted) passing through the fossa ovalis of interatrial septum 33.

Under optional direction of the TEE, a catheter-guiding guide wire 84 is maneuvered into the body through the right jugular vein, through the superior vena cava, across right atrium 12, to enter coronary sinus 52 from cardiac ostium 54.

Negotiation of entrance of the catheter-guiding guide wire 84 from the right atrium 12 to the coronary sinus 52 may be aided, in some embodiments of the invention, by the elongated catheter body 72 including an angle designed to facilitate negotiating the entrance.

Reference is now additionally made to FIG. 5G, which is a schematic depiction of an optional configuration of the first embodiment of the present invention relating to the minimally invasive conduit to the left atrium.

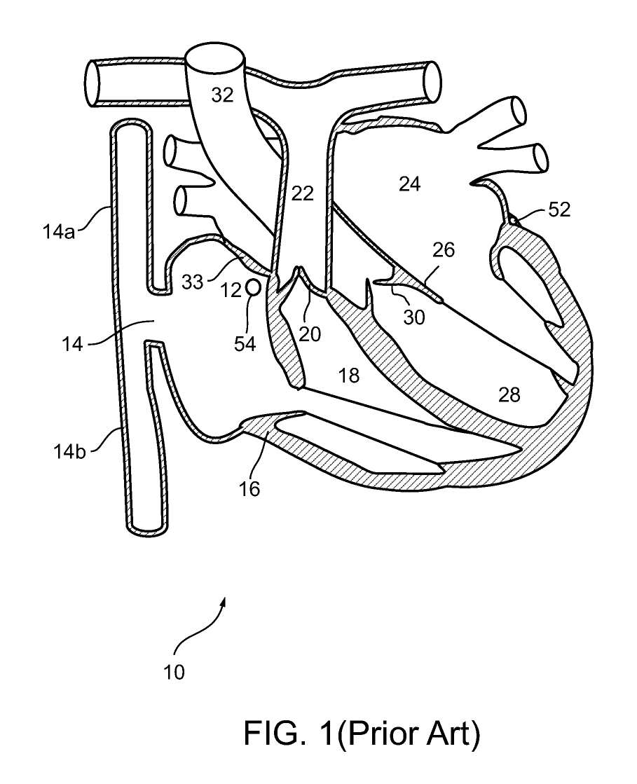

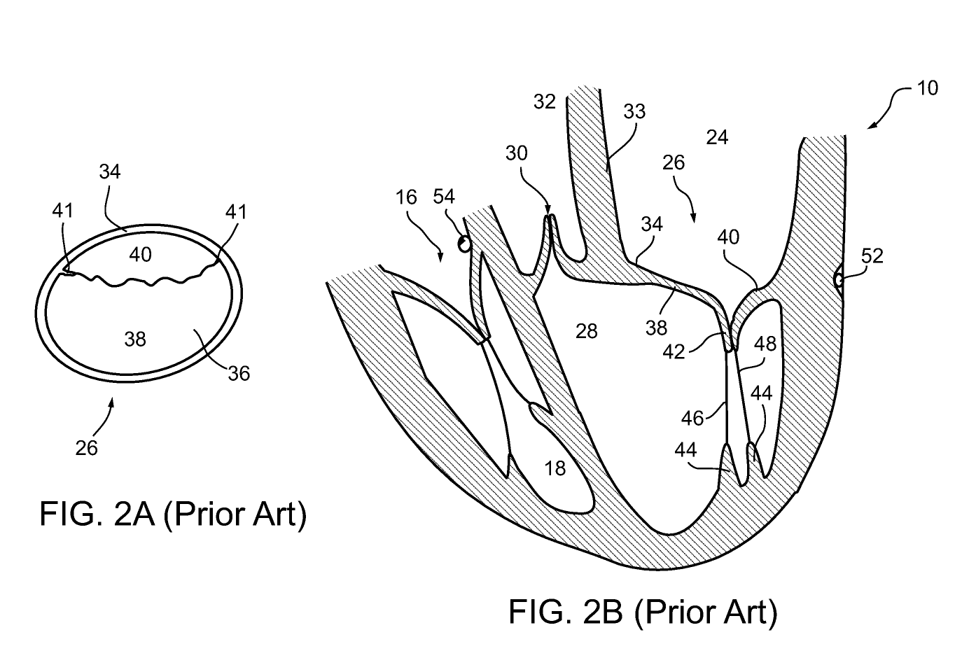

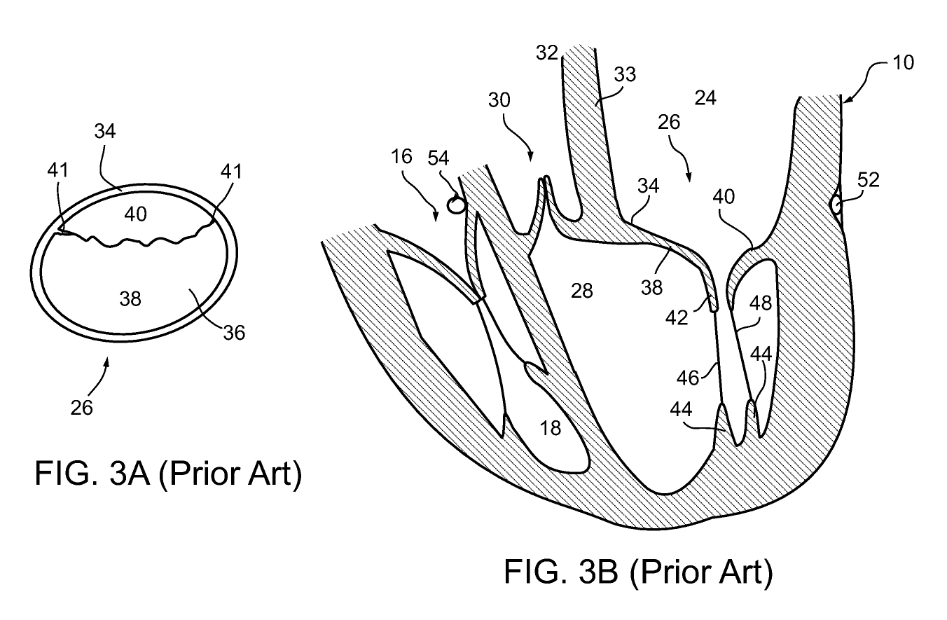

FIG. 5G depicts the elongated catheter body 72, the distal end 76, and the distal tip 78, depicts an angle 205 between the elongated catheter body 72 and the distal end 76. The angle 205 is used to negotiate the entrance to the coronary sinus, which is at an angle from the direction from which the elongated catheter body 72 typically enters the right atrium (12 of FIG. 1). The angle is optionally between about 70° and 110°.

While balloon 90 is in a first non-anchoring state, device 70 is mounted onto catheter-guiding guide wire 84 through catheter-guiding guide wire lumen 88 and directed in the usual way so that distal end 76 of elongated catheter body 72 is located inside coronary sinus 52 where side port 82 faces wall 98 of cardiac tissue separating coronary sinus 52 from left atrium 24.

It is noted that the distal end 76 is optionally of a length and curvature approximating the natural curve of the coronary sinus. Having such as length, and having the curvature naturally preferring an orientation in which the curve conforms to the curve of the coronary sinus, enables the side port 82 to face the wall 98 of cardiac tissue at a correct location, and with the side port 82 correctly oriented toward the wall 98.

Reference is now additionally made to FIG. 5H, which is a schematic depiction of the first embodiment of the present invention relating to the minimally invasive conduit to the left atrium 24.

Balloon 90 is inflated to a second anchoring state, engaging the luminal walls of coronary sinus 52 so as to stabilize and anchor the position of distal end 76 in coronary sinus 52. As also depicted in FIG. 5D, the optionally acircular cross section of balloon 90 does not entirely obstruct the lumen of coronary sinus 52. Further, inflation of balloon 90 pushes side port 82 against wall 98, forming a seal.

A piercing component 100, substantially a 1.5 mm diameter Nitinol wire with a sharpened end is placed into main lumen 80 from proximal end 74 of catheter body 72 and guided through main lumen 80 to emerge out through side port 82, puncturing tissue of wall 98 separating coronary sinus 52 from left atrium 24 to emerge into left atrium 24 above the plane defined by mitral valve annulus 34 above posterior leaflet 40. Optionally, piercing component 100 is subsequently withdrawn from main lumen 80 to leave main lumen 80 free of obstruction.

In such a way, a minimally invasive conduit to left atrium 24 is defined, substantially comprising main lumen 80 of catheter body 74 and the transcoronary sinus puncture made by piercing component 100. In some embodiments, the puncture is cannulated to maintain the conduit entirely open. Since coronary sinus 52 is not entirely obstructed, the conduit may optionally be maintained for an extended period of time (e.g., at least an hour, at least a day, at least a week, and even at least a month).

Through the thus-defined conduit, left atrium 24 is accessible. In some embodiments, an object is passed through at least part of main lumen 80 to proximity of side port 82. In some embodiments, an object is passed through side port 82 and through the puncture into left atrium 24. Any object fitting through the conduit may be passed through the conduit, for example for experimental, diagnostic or therapeutic purposes.

In some embodiments, the object is a medical device, for example a cutting tool, for treating or sampling tissue, for example cardiac tissue, for example from mitral valve 26 or left atrium 24.

In some embodiments, the object is an observation device, for example an optical fiber bundle such as 96, for inspecting tissue, for example cardiac tissue, for example mitral valve 26 or left atrium 24. In some embodiments, the object is a type of sensor, for example a pressure sensor to measure the pressure inside left atrium 24.

In some embodiments, the object is a medical material, such as a fluid, a composition or an active agent, for example an active pharmaceutical ingredient or a composition including an active pharmaceutical ingredient, or a contrast material for imaging devices. In some such embodiments, the passing of the object into left atrium 24 is administration of the object to the patient.

### Reducing Mitral Valve Mobility

In some embodiments, the present invention provides methods and devices related to immobilizing one or more mitral valve leaflets.

In order to simplify understanding of the present invention, an embodiment is discussed hereinbelow in the context of treating a mitral valve suffering from ischemic mitral regurgitation where the teachings of the present invention are directed to performing a “bow-tie” repair of the mitral valve in order to increase mitral valve leaflet coaptation.

“Bow-tie” repair of a mitral valve 26 is discussed above with reference to FIG. 4A. One of the problems encountered when performing such a “bow-tie” repair is that of the proper positioning of the mitral valve leaflet edges to allow deployment of a leaflet-joining device such as suture 60. It is necessary to capture edge 56 of anterior leaflet 38 and edge 58 of posterior leaflet 40, each at a specific location, bring edges 56 and 58 together, and then hold edges 56 and 58 immobile while a leaflet-joining device is deployed.

Capturing and immobilizing leaflets 38 and 40 in a minimally-invasive procedure is no simple task. This challenge is exceptional in cases where leaflet motion is extreme, for example, when there is significant leaflet prolapse into the left atrium or when the leaflets are relatively far apart, as is the case in advanced or severe cases of various mitral valve pathologies, such as severe ischemic mitral regurgitation.

One method of simplifying such capture is to stop the beating of the heart, as suggested in the PCT patent application published as WO 99/00059. One skilled in the art recognizes that stopping the beating of a heart significantly complicates a medical procedure, renders the medical procedure more expensive, decreases the chance of a positive outcome and increases patient mortality.

Due to the difficulties in the capture and immobilization of mitral valve leaflets of a beating heart, minimally-invasive methods of “bow-tie” repair of a mitral valve are time-consuming even when performed by a physician of exceptional skill Often unsatisfactory results are achieved as a leaflet-joining device is deployed near an edge of a leaflet which leads to the risk of leaflet tearing, far from an edge of a leaflet which may lead to insufficient leaflet coaptation or grasps leaflet edges that are skewed which may lead to insufficient coaptation.

This problem is alleviated by some embodiments of the present invention, methods and devices for reducing the mobility of a mitral valve leaflet by passing at least one leaflet-engaging component through cardiac tissue separating the coronary sinus and the left atrium of the heart to enter the left atrium and then engaging at least one mitral valve leaflet to reduce the mobility of that mitral valve leaflet. Subsequently, an additional procedure may be performed, for example deployment of a leaflet-joining device as known in the art.

Thus, some embodiments of the present invention provide for the minimally invasive capture and reduction of mobility of one or more mitral valve leaflets that is quick, simple and accurate. In some embodiments, approaching the mitral valve leaflets by accessing the left atrium from the coronary sinus has one or more advantages over methods for approaching mitral valve leaflets known in the art

In some embodiments, a leaflet-engaging component used in implementing the teachings of the present invention enters the left atrium from the coronary sinus and does not obstruct the approach of other devices to the atrial face of the mitral valve, devices that may access the left atrium by known routes, for example from the right atrium through a transseptal puncture or through the left atrial roof. Thus some embodiments of the present invention are exceptionally suitable for use with known devices. This includes devices useful for direct optical observation of the mitral valve.

In some embodiments of the present invention, a leaflet-engaging component enters the left atrium from the coronary sinus just above the mitral valve annulus (generally proximally to a posterior leaflet). In some embodiments, such entry allows the leaflet-engaging component to move substantially in parallel to the plane defined by the mitral valve annulus, physically limiting the upwards motion of the mitral valve leaflets even before the leaflets are engaged which is exceptionally desirable in cases of mitral valve leaflet prolapse. Additionally, in some embodiments the leaflet-engaging component is necessarily positioned in close proximity to the atrial face of the leaflets by the location of the coronary sinus relative to the mitral valve. In some such embodiments, this simplifies the process of engaging the leaflets. Further, in some embodiments where leaflet engagement is performed with the help of applying suction from the leaflet-engaging component, the leaflet capture process is relatively quick (due to the proximity of a suction port to the surface of the leaflet) so relatively little blood is removed from the heart before the leaflet is engaged, reducing the chance of cardiac exsanguination.

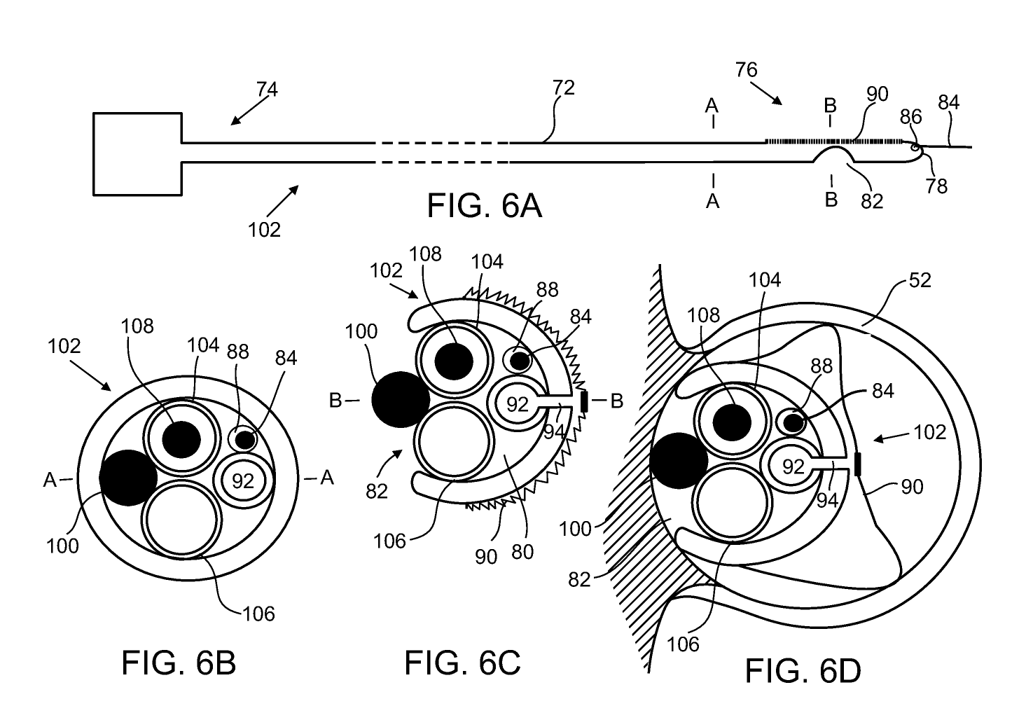

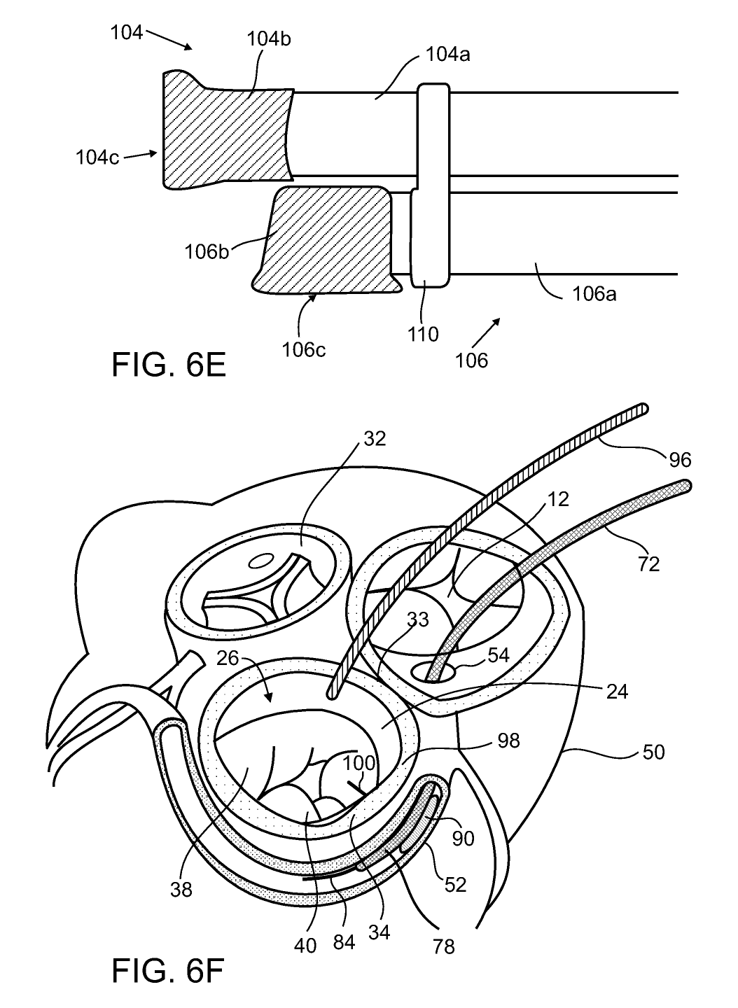

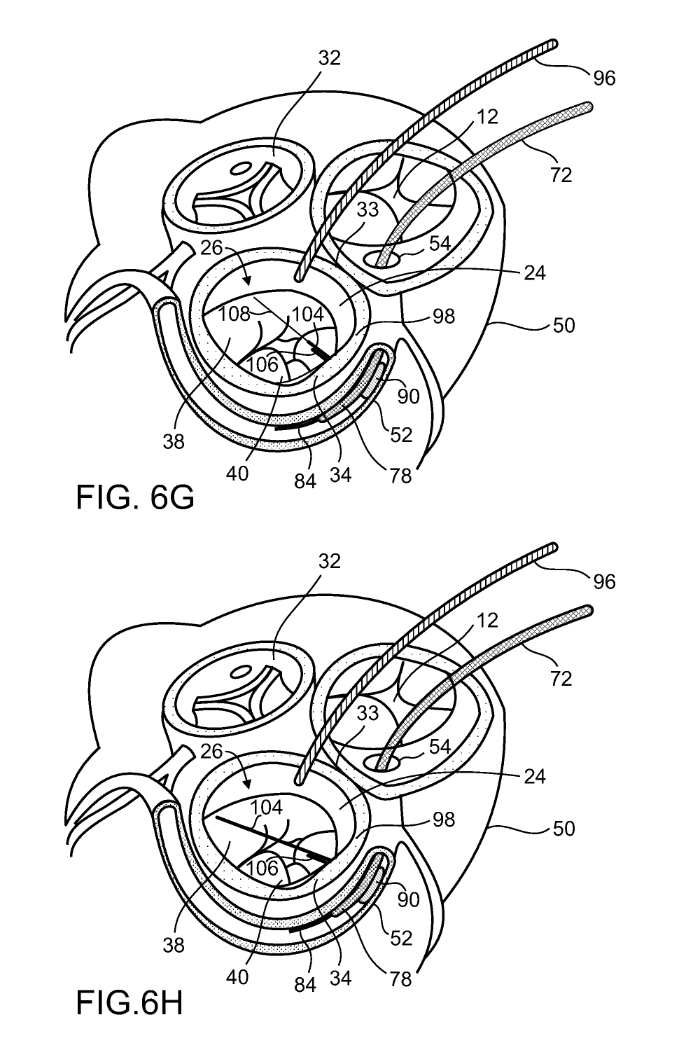

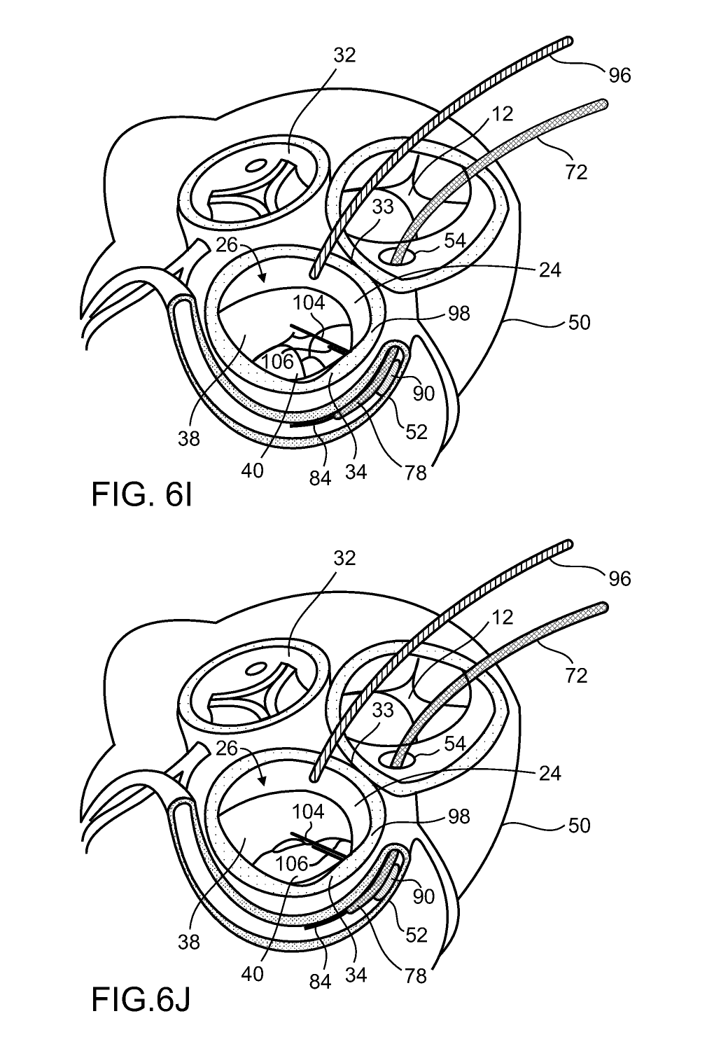

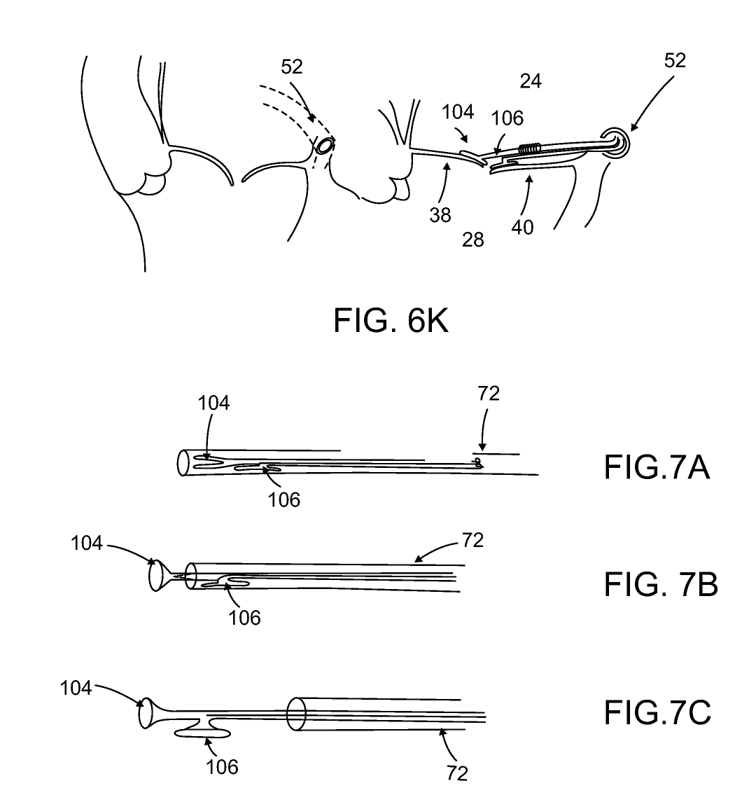

An embodiment of a device for reducing the mobility of a mitral valve leaflet, device 102, is depicted in FIG. 6A (side view), FIG. 6B (cross section A-A) and FIG. 6C (cross section B-B). Device 102 is also depicted deployed in a coronary sinus 52 in FIG. 6D (cross section B-B), FIG. 6E (side view) and FIGS. 6F, 6G, 6H, 6I and 6J (all views including of a heart 50 similar to that in FIG. 5E).

Device 102, like device 70, is substantially a cardiac catheter and includes many of the same components as described above.

Three components of device 102 that do not have equivalents in device 70 are first leaflet-engaging component 104, second leaflet-engaging component 106 and leaflet-engaging component guide wire 108. Before use, device 102 is provided with piercing component 100, first leaflet-engaging component 104, second leaflet-engaging component 106, and leaflet-engaging component guide wire 108 prepacked inside main lumen 80.

In FIG. 6E is seen a detailed side-view depiction of first leaflet-engaging component 104 and second leaflet-engaging component 106 when packed inside main lumen 80. First leaflet-engaging component 104 comprises: i) the distal end of a 1.8 mm pipe 104a that functions both as a first vacuum conduit and as a first leaflet-engaging component director and ii) a soft silicon rubber sleeve 104b defining a first suction port 104c. Second leaflet-engaging component 106 comprises: i) the distal end of a 1.8 mm pipe 106a that functions both as a second vacuum conduit and as a second leaflet-engaging component director and ii) a soft silicon rubber sleeve 106b defining a second suction port 106c. Encircling pipes 104a and 106a is a retainer ring 110 that keeps the pipes close together but allows one to slide past the other. In such a way, first leaflet-engaging component 104 and second leaflet-engaging component 106 are independently maneuverable.

Leaflet-engaging component guide wire 108 passes through the lumen of first leaflet-engaging component pipe 104a.

The proximal end of pipe 104a passes through main lumen 80 to proximal end 74 of catheter body 72 and can be used to move first leaflet-engaging component 104 in and out of main lumen 80 through port 82. Similarly, the proximal end of pipe 106a passes through main lumen 80 to proximal end 74 of catheter body 72 and can be used to move second leaflet-engaging component 106 in and out of main lumen 80 through port 82.

Pipe 104a is functionally associated with a vacuum source (not depicted). When the vacuum source is activated, suction is applied from first suction port 104c through the lumen of pipe 104a. Similarly, the proximal end of pipe 106a is functionally associated with a vacuum source (not depicted). When the vacuum source is activated, suction is applied from second suction port 106c through the lumen of proximal end of pipe 106a. A vacuum actuator (not depicted) is functionally associated with the vacuum source or sources and is configured to activate, deactivate and vary the suction applied at first suction port 104c and second suction port 106c independently.

An embodiment of a method for limiting the mobility of a mitral valve leaflet is discussed in detail with reference to device 102 FIGS. 6A, 6B, 6C, 6D, 6E and especially FIGS. 6F, 6G, 6H, 6I, 6J and 6K.

Distal end 76 of elongated catheter body 72 of device 102 is directed into a coronary sinus 52 of heart 50 with the help of catheter-guiding guide wire 84. Balloon 90 inflated to a second anchoring state to stabilize the position of distal end 76 and side port 82 in coronary sinus 52 as described above for device 70 and depicted in FIG. 6D.

Piercing component 100 is advanced through side port 82, puncturing the tissue making up wall 98 separating coronary sinus 52 from left atrium 24 to emerge into left atrium 24 above the plane defined by mitral valve annulus 34 above posterior leaflet 40, FIG. 6F. Piercing component 100 is withdrawn from main lumen 80.

In FIG. 6G, leaflet-engaging component guide wire 108, first leaflet-engaging component 104 and second leaflet-engaging component 106 are advanced together through the tissue of wall between coronary sinus 52 and left atrium 24 through the puncture made by piercing component 100 to enter left atrium 24 by manipulating pipes 104a and 106a.

In FIG. 6H, under optical guidance through optical fiber bundle 96, first leaflet-engaging component 104 is advanced through left atrium 24, substantially in parallel to the plane defined by mitral valve annulus 34 first above posterior leaflet 40 and then above anterior leaflet 38. It is important to note that throughout, heart 50 is beating and leaflets 38 and 40 move up and down. When the distal end of first leaflet-engaging component 104 is close to an atrial face of anterior leaflet 38, leaflet-engaging component guide wire 108 is withdrawn and first leaflet-engaging component 104 functionally associated with a vacuum source. The vacuum actuator is used to generate suction through first suction port 104c of first leaflet-engaging component 104. The suction causes first leaflet-engaging component 104 to engage an atrial face of anterior leaflet 38 (as opposed to an edge of anterior leaflet 38). A result of first leaflet-engaging component 104 engaging anterior leaflet 38 is that the mobility of anterior leaflet 38 is limited.

In FIG. 6I, while suction is applied through first suction port 104c, first leaflet-engaging component 104 is pulled back into side port 82. As a result, first suction port 104c slides across the upper surface of anterior leaflet 38 towards the luminal edge of anterior leaflet 38. As first suction port 104c is progressively pulled towards the luminal edge of anterior leaflet 38, the mobility of anterior leaflet 38 is increasingly limited and the luminal edge of anterior leaflet 38 is lifted upwards towards left atrium 24.

In FIG. 6J, the vacuum actuator is used to begin suction through second suction port 106c of second leaflet-engaging component 106. The applied suction causes second leaflet-engaging component 106 to engage an atrial face of posterior leaflet 40 (as opposed to an edge of posterior leaflet 40). A result of second leaflet-engaging component 106 engaging posterior leaflet 40 is that the mobility of posterior leaflet 40 is limited. First leaflet-engaging component 104 and second leaflet-engaging component 106 are moved until the edges of anterior leaflet 38 and posterior leaflet 40 are close together (out of the view of FIG. 6J as leaflets 38 and 40 coapt in the right ventricle) and are limited in mobility.

Reference is now additionally made to FIG. 6K, which is a schematic depiction of the embodiment of FIGS. 6A-6J, after the mitral valve leaflets 38 40 have been engaged by the leaflet-engaging components 104 106.

The first leaflet-engaging component 104 and the second leaflet-engaging component 106 are depicted having entered the left atrium 24 from the coronary sinus 52. The first leaflet-engaging component 104 has engaged the anterior leaflet 38, and the second leaflet-engaging component 106 has engaged the posterior leaflet 40. Optionally, the first leaflet-engaging component 104 has slid along a face of the anterior leaflet 38 in order to achieve the engagement of the anterior leaflet 38 depicted in FIG. 6K. Optionally, the second leaflet-engaging component 106 has slid along a face of the posterior leaflet 40 in order to achieve the engagement of the posterior leaflet 40 depicted in FIG. 6K.

It is noted that the 104 and the 106 can optionally slide together, and/or separately from each other, as described above with reference to FIG. 6E.

Once the mobility of anterior mitral valve leaflet 38 and posterior mitral valve 40 is limited as described above by engagement with first leaflet-engaging component 104 and second leaflet-engaging component 106, respectively, a desired additional intervention may be performed. For example, in some embodiments, an additional device is directed into heart 50 to proximity with mitral valve or mitral valve leaflets 38 and 40, for example to treat mitral valve 26 or mitral valve leaflet 38 and/or 40.

It is noted that the device 102 of FIGS. 6A-6D may optionally include one suction port, two suction ports, or more suction ports, rubber sleeves, and tubes such as the suction ports 104c 106c, the rubber sleeves 104b 106b, and tubes 104a 106a of FIG. 6E. Each of the suction ports may be maneuvered separately, and or some or all may be maneuvered together. It is noted that the retainer ring 110 enables optional maneuvering together, while also allowing one of the suction ports to slide past another.

Reference is now additionally made to FIGS. 7A, 7B, and 7C, which are schematic depictions of part of an alternative embodiment of FIGS. 6A-6K, at various extensions from an elongated catheter body 72.

FIG. 7A depicts a first leaflet-engaging component 214, and a second leaflet-engaging component 216, within the elongated catheter body 72.

The first leaflet-engaging component 214 is similar to the first leaflet-engaging component 104 of FIG. 6E, yet has a more pronounced suction-cup. The second leaflet-engaging component 216 is similar to the second leaflet-engaging component 106 of FIG. 6E, and also has a more pronounced suction-cup.

FIG. 7B depicts the first leaflet-engaging component 214 extended out from the elongated catheter body 72, while the second leaflet-engaging component 216 is still within the elongated catheter body 72. In this depiction the first leaflet-engaging component 214 is operational to engage the anterior leaflet 38.

FIG. 7C depicts the first leaflet-engaging component 214 extended out from the elongated catheter body 72, and the second leaflet-engaging component 216 also extended out from the elongated catheter body 72. In this depiction the second leaflet-engaging component 216 is also operational, to engage the anterior leaflet 38. In fact, in this depiction both leaflet-engaging components 214 216 are operational to engage the leaflets 38 40.

Once one or both of the leaflets 38 40 are engaged, various operations described herein may optionally be performed.

By way of a non-limiting example, one or both of the leaflets 38 40 may be allowed to move with the beating heart for some time; one or both of the leaflets 38 40 may be pulled so as to restrict movement of the leaflets; and one of the leaflets 38 40 may be allowed to move while the other one of the leaflets 38 40 is restricted in movement.

Some example operations which are optionally performed while one or both of the leaflets 38 40 are engaged according to an embodiment of the invention are a valve relocation operation such as described in PCT Patent Application WO 2007/138572, filed on 21 May 2007 by the inventor of the present invention; and a leaflet enhancement operation such as described in PCT Patent Application PCT/IL2008/000758, filed on 4 Jun. 2008, based on US Provisional Patent Application No. 60/924,869, filed on 2 Dec. 2007, the contents of which are incorporated by reference as if fully set forth herein.

An additional example operation which is optionally performed while one or both of the leaflets 38 40 are engaged according to an embodiment of the invention is the above-mentioned percutaneous suture leaflet repair system named Mobius. The Mobius system involves delivering sutures across the mitral valve leaflets, edge-to-edge, using an Alfieri approach, and securing the sutures with a Nitinol clip. In a clinical trial named Milano II, which did not use the present invention, 6 out of 15 stitches applied did not hold after 30 days. Engaging the leaflets 38 40 according to an embodiment of the invention should improve the stitching and decrease the rate of non-holding stitches.

An additional example operation which is optionally performed while one or both of the leaflets 38 40 are engaged according to an embodiment of the invention is the operation for attaching an implant that is sized and configured to attach in, on, or near the annulus of a dysfunctional heart valve. In use, the implant extends either across the minor axis of the annulus, or across the major axis of the annulus, or both. The implant restores to the heart valve annulus and leaflets a more functional anatomic shape and tension. The more functional anatomic shape and tension are conducive to coaptation of the leaflets, which, in turn, reduces retrograde flow or regurgitation. The device is described in US Published Patent Application 2004/0260393 of Randert et al.

An additional example operation which is optionally performed via an approach through the coronary sinus according to an embodiment of the invention is an improvement on the operation described in US Published Patent Application 2008/091264 of Machold et al. The above-mentioned patent application describes implants or systems of implants and methods which apply a selected force vector or a selected combination of force vectors within or across the left atrium, which allow mitral valve leaflets to better coapt. The implants or systems of implants and methods may also utilize a bridge stop to secure the implant, and the methods of implantation employ various tools. According to an embodiment of the present invention the approaches taught by Machold et al., which require two catheters coming through two different pathways into the left atrium, are combined in the method of this invention, coming in through one pathway and through the coronary sinus.

It is noted that entrance of the leaflet engaging components 214 216 through the coronary sinus 52 and the wall of the left atrium 24 places the leaflet engaging components 214 216 at a convenient location for accessing the leaflets of the mitral valve (see FIG. 6K). Entering via the coronary sinus 52 aids finding the leaflets 38 40, and aids sliding along the atrial wall and along the leaflets 38 40.

In some embodiments, one or more additional devices are directed to a desired location in proximity of mitral valve 26 via any route that is known to one skilled in the art.

In some embodiments, an additional device is directed to the desired location from the ventricular side of mitral valve 26, for example via the cardiac apex from the thoracic cavity or via aorta 32, where aorta 32 is accessed, for example, via a subclavian, carotid or femoral artery, for example using a retrograde approach.

In some embodiments, an additional device is directed to a desired location from the atrial side of mitral valve 26, for example via the roof of left atrium 24 from the thoracic cavity or using a percutaneous antegrade approach with a transseptal puncture, generally through the fossa ovalis.

For example, in some embodiments the additional device is a mitral valve leaflet-capturing device used to deploy a leaflet connecting device to join the edges of anterior leaflet 38 and posterior leaflet 40 in order to perform a variant of the Alfieri “bow-tie” repair of mitral valve 26.

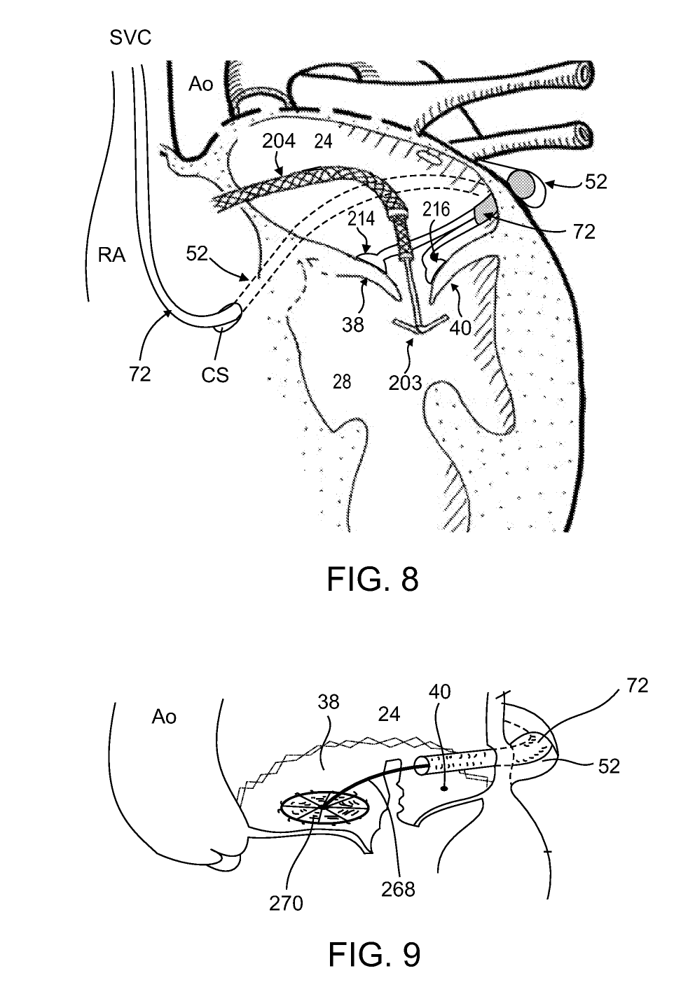

Reference is now additionally made to FIG. 8, which is a schematic depiction of the embodiment of FIGS. 6A-6K and 7, in use for reducing the mobility of mitral valve leaflets while a mitral valve procedure is being performed.

FIG. 8 depicts an elongated catheter body 72 passing through the coronary sinus 52; a first leaflet-engaging component 214 engaging an anterior leaflet 38; and a second leaflet-engaging component 216 engaging a posterior leaflet 40, as described above with reference to FIGS. 6A-6K.

A catheter 204 is inserted into the left atrium 24, and a therapy device 203 is advanced beyond the catheter 204, through the mitral valve 26, into the left ventricle 28, as described above with reference to FIG. 4B.

Exemplary useable leaflet-capturing devices include devices disclosed in PCT patent application published as WO 99/00059; Europe patent application EP 1674040 and U.S. Pat. Nos. 6,165,183; 6,575,971; 6,719,767 and 6,752,813 which approach mitral valve leaflets 38 and 40 substantially perpendicularly to the plane defined by mitral valve annulus 34.

Exemplary leaflet-connecting devices deployed in order to join the edges of the mitral valve leaflets include devices (e.g., clips, sutures, rings) disclosed in the PCT patent application published as WO 99/00059; EP patent application EP 1674040 and U.S. Pat. Nos. 6,165,183; 6,575,971; 6,719,767 and 6,752,813.

The leaflet-capturing devices are used and the leaflet-connecting devices are deployed substantially as described in the relevant reference, but due to the reduction of the mobility of the mitral valve leaflets as described above and specifically engagement of anterior leaflet 38 by first leaflet-engaging component 104 and posterior leaflet 40 by second leaflet-engaging component 106, the capture and alignment of the leaflets is relatively simple, quick and accurate.

The embodiment of the method for reducing leaflet mobility discussed above is described with reference to facilitating “bow-tie” repair of a mitral valve.

In some embodiments, the reduction of leaflet mobility is used for facilitating other procedures, for example, mitral valve leaflet augmentation such as described in U.S. Provisional Patent Application 60/924,869 of the Inventor, the contents of which are hereby incorporated by reference.

Reference is now additionally made to FIG. 9, which is a schematic depiction of a third embodiment of the present invention relating to mitral valve leaflet augmentation.

As described above with reference to other embodiments, an elongated catheter body 72 enters the left atrium 24 through the coronary sinus 52. In the embodiment of FIG. 9, the elongated catheter body 72 is used to deploy an obstructor deployment catheter 268. The obstructor deployment catheter 268 deploys an expandable obstructor 270, in the anterior leaflet 38.

In the embodiment discussed above, guidance of leaflet-engaging components 104 and 106 is performed with the help of optical observation through optical fiber bundle 96 that passes through the body of the subject including past the femoral vein, the inferior vena cava, right atrium 12 and into left atrium 24 through a transseptal puncture. In some embodiments, an optical fiber or similar component is positioned to guide leaflet-engaging components through a different path through the body of the subject. In some embodiments, a different guiding modality, such as fluoroscopy, echocardiography, intravascular ultrasound, angioscopy, magnetic-resonance imaging, radiographic imaging or other ultrasound imaging is used to assist in guiding leaflet-engaging components. In embodiments, the same imaging modality that is used to direct a distal end of an elongated catheter body into a coronary sinus is also used to assist in guiding leaflet-engaging components to engage one or both mitral valve leaflets.

In the embodiment of the method for reducing leaflet mobility described above, anterior leaflet 38 is engaged by first leaflet-engaging component 104 and posterior leaflet 40 by second leaflet-engaging component 106. In some embodiments, an anterior mitral valve leaflet is engaged by a second leaflet-engaging component and a posterior mitral valve leaflet is engaged by a first leaflet-engaging component.

In the embodiment of the method for reducing leaflet mobility described above, two leaflet-engaging components 104 and 106 were directed from the coronary sinus 52 into left atrium 24 to engage leaflets 38 and 40.

In some embodiments, only a single leaflet (whether anterior leaflet 38 or posterior leaflet 40) is engaged by a leaflet-engaging component from a coronary sinus. In some embodiments, a procedure or intervention is performed when the mobility of only one mitral valve leaflet is reduced.

In some embodiments, a procedure or intervention is performed when the mobility of only one leaflet (whether the anterior or posterior leaflet) is reduced by a leaflet-engaging component from a coronary sinus while the mobility of the second leaflet is reduced by engaging the second mitral valve leaflet with a second leaflet-engaging device. In some embodiments, the second leaflet-engaging device approaches the second leaflet from a direction substantially perpendicular to the plane defined by the annulus of the mitral valve. Such an additional device may be directed to an appropriate location for engaging the second leaflet via any route that is known to one skilled in the art.

In some embodiments, the second leaflet-engaging device approaches the second leaflet through the left ventricle. For example, in some embodiments, the second leaflet-engaging device enters the left ventricle by passing through an aorta of the heart into the left ventricle (e.g., a percutaneous retrograde approach, entering via a subclavian, carotid or femoral artery). In some embodiments, the second leaflet-engaging device enters the left ventricle for example through the thoracic cavity passing through the cardiac apex into the left ventricle.

In some embodiments, the second leaflet-engaging device approaches the second leaflet through the left atrium. For example, in some embodiments, the second leaflet-engaging device enters the left atrium by passing through an intraatrial septum (e.g., via a transseptal puncture through the fossa ovalis) from the right atrium into the left atrium (e.g. a percutaneous antegrade approach with a transseptal puncture, generally through the fossa ovalis). For example, in some embodiments, the second leaflet-engaging device enters the left atrium by passing through a roof of the left atrium from the thoracic cavity.

In some embodiments, the posterior leaflet is engaged by a first leaflet-engaging component entering the left atrium from the coronary sinus and the anterior leaflet is engaged by the second leaflet-engaging device.

In some embodiments, the anterior leaflet is engaged by a first leaflet-engaging component entering the left atrium from the coronary sinus and the posterior leaflet is engaged by the second leaflet-engaging device.

In some embodiments, the anterior leaflet is engaged by a first leaflet-engaging component entering the left atrium from the coronary sinus and the posterior leaflet is engaged by a second leaflet-engaging device entering the left atrium through the interatrial septum. In some embodiments, such a “cross over” is preferred as the second leaflet-engaging device describes a relatively straight path.

In the embodiment described above, device 102 comprises two leaflet-engaging components 104 and 106. In some embodiments, a device comprises only a single leaflet-engaging component or more than two leaflet-engaging components. In some embodiments, when a device having a single leaflet-engaging component is used, the leaflet-engaging component is used to engage a posterior leaflet 40 in order to reduce the mobility of that leaflet. In some embodiments, when a device having a single leaflet-engaging component is used, the leaflet-engaging component is used to engage an anterior leaflet 38 in order to reduce the mobility of that leaflet. In some such embodiments, a corresponding leaflet-engaging component director (e.g., a rod or the like, analogous to pipes 104a and 106a of device 102) is used to advance the single leaflet-engaging component across the atrial face of the posterior leaflet towards the anterior leaflet substantially in parallel to the plane defined by the mitral valve annulus, to approach and engage the anterior leaflet at a location on the atrial face of the anterior leaflet. Once the leaflet-engaging component engages the anterior leaflet, the mobility of the posterior leaflet is reduced due to the presence of the leaflet-engaging component director laying across the atrial face of the posterior leaflet. Such a situation is analogous to the depicted in FIGS. 6H and 6I, where pipe 104a lays across the atrial face of posterior leaflet 40 when engaging anterior leaflet 38.

In device 102 described above, leaflet-engaging components 104 and 106 engage leaflets 38 and 40 by the application of suction through suction ports 104c and 106c, respectively. In some embodiments, one or both leaflets are engaged with other types of leaflet-engaging components, for example, physical means such as clamps, pincers, hooks, barbs or other piercing components.

In device 102 described above, leaflet-engaging components 104 and 106 are guided to a desired location on the atrial face of leaflets 38 and 40 with the help of leaflet-engaging component guide wire 108 that passes through the lumen of first vacuum conduit 104a. In some embodiments, a leaflet-engaging component guide wire passes through an elongated catheter body in a dedicated leaflet-engaging component guide wire lumen. In some embodiments, a leaflet-engaging component guide wire passes through a non-dedicated lumen of an elongated catheter body, such as main lumen 80. In some embodiments, a device of the present invention is devoid of a leaflet-engaging component guide wire and the leaflet-engaging components are configured for guidance to a desired location without the use of a guide wire. In some embodiments, each leaflet-engaging component is provided with a dedicated a leaflet-engaging component guide wire.

In the embodiments described above, the anchoring component, inflatable balloon 90 is configured to avoid complete obstruction of the lumen of a coronary sinus 52 in which deployed when in the second-anchoring state by having an acircular cross-section when inflated, specifically, a bidentate cross-section. In some embodiments, an anchoring component has a different acircular cross-section. In some embodiments, an anchoring component comprises a component other than an inflatable balloon, for example extendible/retractable arms, a stent-like expandable tube and the like. In some embodiments, an anchoring component is configured to substantially completely obstruct a coronary sinus when in a second anchoring state.

In the embodiments of the devices described above, there is a single side port configured to provide access to the left atrium, port 82 located on the side of elongated catheter body 72 in communication with main lumen 80. In device 102, both first leaflet-engaging component 104 and second leaflet-engaging component 106 are configured to move in and out of main lumen 80 through side port 82. In some embodiments, a device is provided with more than one side port. For example, in some embodiments of a device for limiting leaflet mobility, there is a port for each leaflet-engaging component or there is at least one additional port used for other components, e.g. an interventional tool or an observation tool. For example, in some embodiments of a device suitable for defining a minimally-invasive conduit, there is more than one side port, allowing passing of one or more objects from one or more different directions into a left atrium.

In the embodiments of the devices described above, there is a single main lumen 80 passing from a proximal end 74 to a side port 82 through elongated catheter body 72. Consequently, a single discrete conduit is defined when device 70 is deployed inside a coronary sinus while in device 102, both leaflet-engaging components pass through the same main lumen 80. In some embodiments, a device is provided with more than one main lumen. In some embodiments, each such lumen passes from a proximal end to the same port near the distal end of an elongated catheter body. In some embodiments, each such lumen passes from a proximal end to terminate at a separate port near the distal end of an elongated catheter body. For example, in some embodiments of a device for limiting leaflet mobility, there is a separate lumen through which a director of a leaflet-engaging component passes, or a separate lumen for each leaflet-engaging component. For example, in some embodiments of a device suitable for defining a minimally-invasive conduit, there is more than one main lumen each terminating at a separate side port near the distal end of an elongated catheter body.

In some embodiments, an elongated catheter body of a device is provided with more than two such ports. For example, in some embodiments of a device suitable for acting as a conduit to the left atrium, there are two separate side ports allowing access to the left atrium from different angles. For example, in some embodiments of a device for limiting leaflet mobility, there is a separate port for each leaflet-engaging component, so that each leaflet-engaging component is configured to move in and out of a lumen in the catheter body through a different port. Generally, but not necessarily, when such devices are used it is necessary to make a separate coronary sinus puncture in proximity of each port through which a corresponding leaflet-engaging component is directed into the left atrium. In some embodiments, each port is associated with a different lumen.

Leaflet-engaging device 102 is provided with a port 82 disposed on the side of catheter body 72 from which both leaflet-engaging components 104 and 106 emerge. In some embodiments, a leaflet-engaging device is provided with a port that is not disposed at the side of the catheter body. For example, in some embodiments, one or more leaflet-engaging components emerge from a port disposed at the tip of a catheter body.

In the embodiments of the device described above, piercing component 100 is a discrete elongated component having substantially only one function: to make a coronary sinus puncture to allow passage of objects into the left atrium. In some embodiments of the invention, a piercing component has a different shape or configuration, is not discrete, is integrally formed with another component or serves an additional function. For example, in embodiments of a device useful for reducing leaflet mobility, one or more leaflet-engaging components or a leaflet-engaging component guide wire is configured to function as a piercing component.

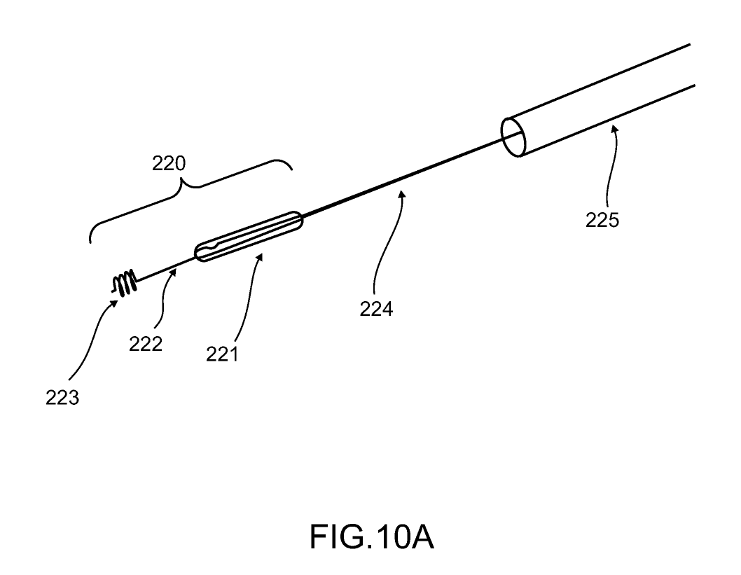

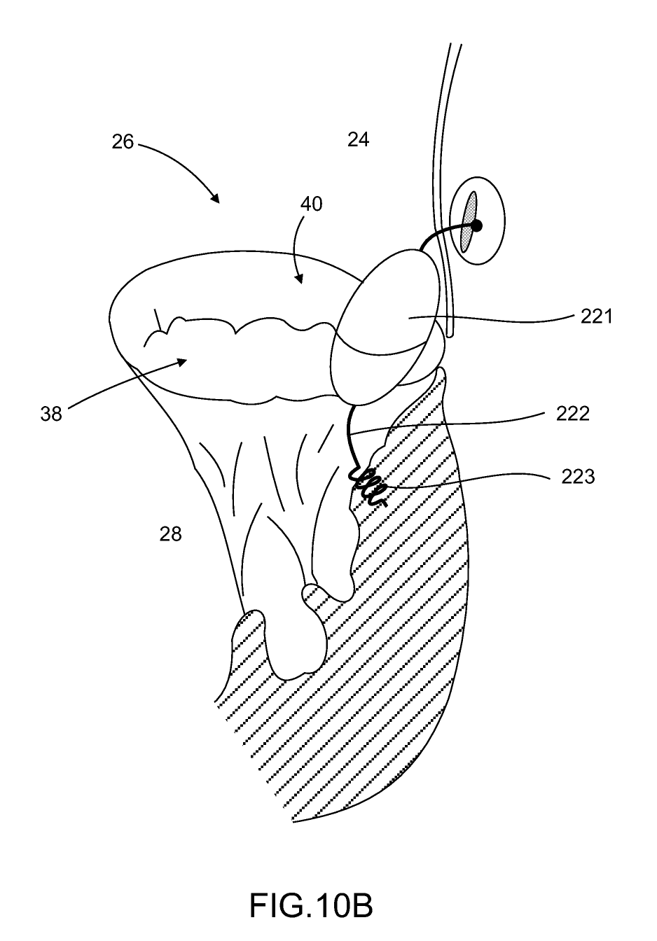

Reference is now additionally made to FIG. 10A, which is a schematic depiction of a fourth embodiment of the present invention relating to a mitral valve obstruction device.

The mitral valve anti-regurgitation device 220 includes an obstruction 221 connected to an extension wire 222, which is further connected to an anchor 223.

The anti-regurgitation device 220 passes through a catheter 225, at the end of a wire 224.

Reference is now additionally made to FIG. 10B, which is a schematic depiction of the fourth embodiment of FIG. 10A, deployed in a heart.

The anchor 223 is depicted as having been attached to a wall of the left ventricle 28. By way of a non-limiting example, the anchor 223 is optionally a sharp wire with a shape of a corkscrew, suitable for anchoring in the wall of the left ventricle. By way of another non-limiting example, the anchor 223 is optionally a staple, suitable for anchoring in the wall of the left ventricle.

The obstruction 221 of the anti-regurgitation device 220 is connected to the anchor 223 by the extension wire 222, placing the obstruction 221 between the anterior leaflet 38 and the posterior leaflet 40 of the mitral valve 26.

The obstruction 221 is connected to the wire 224 which anchors the anti-regurgitation device 220 to a wall of the left atrium 24. The anchoring to the wall of the left atrium is optionally done at the hole between the left atrium 24 and the coronary sinus, which heals around the wire 224. An alternative optional anchoring is performed by passing the wire 224 back through the hole between the left atrium 24 and the coronary sinus, and anchoring the wire 224 within the coronary sinus, and not at the hole.

The obstruction 221 is therefore optionally anchored to the heart at two points. It is noted that the obstruction 221 may optionally be anchored at only one point.

The obstruction 221 fills a gap which exists in hearts having mitral valve prolapse, between the anterior leaflet 38 and the posterior leaflet 40 of the mitral valve 26. The anterior leaflet 38 and the posterior leaflet 40 press against the obstruction 221, leaving no room for mitral regurgitation.

The obstruction 221 acts as a filler for one or both leaflets to close on. The obstruction 221 can extend both above and below the mitral valve 26. The obstruction 221 can be curved, for example when attached to a wall. The obstruction 221 optionally has a round cross-section, in which case there is no need to control orientation of the obstruction 221. The obstruction 221 optionally has a uniform cross-section. The obstruction 221 is optionally inflatable. The obstruction 221 is optionally expandable.

In some embodiments, various components of a device are provided with markers opaque to a desired guiding modality allowing for more accurate direction and deployment of the device. For example, in embodiments, a port such as a side port 82 or an inflation port 94, is marked at or around the periphery of the port. Typical guiding modalities include fluoroscopy, echocardiography, intravascular ultrasound, angioscopy, magnetic-resonance imaging, radiographic imaging or other ultrasound imaging modalities.

Devices of the present invention may be fashioned from commercially available components or using standard techniques and standard materials known in the art and described, for example, in the cited art. Suitable materials are in general biocompatible non-immunogenic materials including metals such as titanium, Nitinol, stainless steel and polymeric materials such as polyethylene, polyurethane, polysulfone, polyetheretherketone (PEEK) and silicon rubber. Some embodiments of the present invention are provided with coatings, for example, polyfluorinated hydrocarbons such as polytetrafluorethylene or antimicrobial coatings (e.g., including silver metal or silver ion coatings).

In the embodiments described above, leaflet-engaging device 102 and conduit defining device 70 are configured to enter the cardiovascular system from the right jugular vein, to pass through a superior vena cava, a right ventricle and to enter a coronary sinus through a cardiac ostium.

In some embodiments, a device and particularly an elongated catheter body thereof is configured to enter the cardiovascular system from a different point and to pass a different path into a coronary sinus.

For example, in some embodiments, a device is configured to enter the cardiovascular system from the left jugular vein to pass through the superior vena cava, the right ventricle and to enter the coronary sinus through the cardiac ostium.

For example, in some embodiments, a device is configured to enter the cardiovascular system from a subclavian vein (in some embodiments the left, in some embodiments the right subclavian vein) to pass through the superior vena cava, the right ventricle and to enter the coronary sinus through the cardiac ostium.

For example, in some embodiments, a device is configured to enter the cardiovascular system from a femoral vein (in some embodiments the left, in some embodiments the right femoral vein) to pass through the inferior vena cava, the right ventricle and to enter the coronary sinus through the cardiac ostium.

For example, in some embodiments, a device is configured to enter the body from an incision in the thorax into the thoracic cavity and to enter the coronary sinus through the wall of the coronary sinus.

It is generally preferred to use a device to enter a coronary sinus in accordance with its designed configuration. That said, in some embodiments a device particularly configured for passage through one path is directed into the coronary sinus via another path. In some embodiments, a device is configured for more general use, and is configured for passage into the coronary sinus via two or more paths.

In the embodiments described above, a distal end 76 of a catheter body 72 of a device 70 or 102 is placed into the right jugular vein of a human subject, and from there directed through the superior vena cava, past left atrium 12, to enter coronary sinus 52 from cardiac ostium 54. In some embodiments, such an approach is preferable due to the fact that such a path is relatively short, relatively direct and because the jugular veins are of a relatively large diameter.

In some embodiments of the method of the present invention, a distal end of an elongated catheter body of a device is directed into a coronary sinus from a different entry point or through a different path. One skilled in the art is able to implement the method of the present invention with reference to the description herein with reference to the art describing minimally-invasive access of catheters into a coronary sinus of a mammalian heart, see for example, U.S. Pat. No. 6,790,231 and U.S. Pat. No. 7,004,958.

For example, in some embodiments, a distal end of an elongated catheter body enters the cardiovascular system from the left jugular vein and is directed through the superior vena cava and the right ventricle to enter the coronary sinus through the cardiac ostium.

For example, in some embodiments, a distal end of an elongated catheter body enters the cardiovascular system from a subclavian vein (in some embodiments the left, in some embodiments the right subclavian vein) and is directed through the superior vena cava and the right ventricle to enter the coronary sinus through the cardiac ostium.

For example, in some embodiments, a distal end of an elongated catheter body enters the cardiovascular system from a femoral vein (in some embodiments the left, in some embodiments the right femoral vein) and is directed through the inferior vena cava and the right ventricle to enter the coronary sinus through the cardiac ostium.

For example, in some embodiments, a distal end of an elongated catheter body enters the cardiovascular system through an incision in the thorax and is directed thorax into the thoracic cavity and to enter the coronary sinus through the wall of the coronary sinus.

In the embodiments described above, heart 50 beats while the method if implemented. In some embodiments, the heart is stopped, e.g., induced cardioplegia.

Embodiments of the present invention have been described herein primarily with reference to treatment of living human subjects. It is understood, however, that embodiments of the present invention are performed for the veterinary treatment of a non-human mammal, especially horses, cats, dogs, cows, sheep and pigs.

Embodiments of the present invention have been described herein primarily with reference to treatment of living subjects. It is understood that application of the present invention for training and educational purposes (as opposed to treating a condition) falls within the scope of the claims, whether on a living non-human subject or on a dead subject, whether on a simulated human body, a human cadaver or on a non-human body, whether on a valve in a heart isolated (at least partially) from a body, or on a body.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the present invention.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for engaging mitral valve leaflets, comprising:
a) directing a distal end of an elongated catheter body of a leaflet-engaging device into a coronary sinus of a heart;
b) passing a first leaflet-engaging component located proximate to said distal end of said elongated catheter body of said leaflet-engaging device through cardiac tissue separating said coronary sinus and a left atrium of said heart to enter a left atrium of said heart; and
c) engaging a first mitral valve leaflet with said first leaflet-engaging component,
thereby engaging at least one mitral valve leaflet.

2. (canceled)

3. The method of claim 1, wherein said first leaflet-engaging component engages said mitral valve leaflet at an atrial face of said first mitral valve leaflet.

4. (canceled)

5. The method of claim 1, wherein said engaging of said first mitral valve leaflet comprises application of suction through said first leaflet-engaging component, and further comprising said first leaflet-engaging component sliding along a face of said first mitral valve leaflet.

6-7. (canceled)

8. The method of claim 1, further comprising engaging a second mitral valve leaflet.

9-10. (canceled)

11. The method of claim 8, wherein said engaging of said second mitral valve leaflet is with a second leaflet-engaging component located proximal to said distal end of said leaflet-engaging device, and wherein said second leaflet-engaging component engages said second mitral valve leaflet at an atrial face of said second mitral valve leaflet.

12-14. (canceled)

15. The method of claim 11, wherein said first leaflet-engaging component and said second leaflet-engaging component of said leaflet-engaging device are independently maneuverable, and further comprising: moving said first leaflet-engaging component and said second leaflet-engaging component one relative to the other so as to change the relative orientation of said engaged first leaflet and said engaged second leaflet.

16-20. (canceled)

21. The method of claim 1, further comprising:
directing a second leaflet-engaging device to proximity of a second mitral valve leaflet; and
engaging a second mitral valve leaflet with said second leaflet-engaging device.
wherein said directing of said second leaflet-engaging device to proximity of said second mitral valve leaflet is through a left ventricle of said heart.

22. The method of claim 1, further comprising:
directing a second leaflet-engaging device to proximity of a second mitral valve leaflet; and
engaging a second mitral valve leaflet with said second leaflet-engaging device,
wherein said directing of said second leaflet-engaging device to proximity of said second mitral valve leaflet is through a left atrium of said heart.

23-26. (canceled)

27. A device for engaging cardiac valve leaflets, comprising:
a) an elongated catheter body with a proximal end and a distal end, said distal end shaped for passage into a heart of a mammalian body; and
b) a first leaflet-engaging component located proximate to said distal end of said catheter body.

28. The device of claim 27 in which said first leaflet-engaging component comprises a suction device.

29. The device of claim 27 in which said distal end is at an angle of between about 70° to 110° to a longitudinal axis of said elongated catheter body, said angle located proximally to said distal end, and in which said angle is located at about 5 centimeters +/−20% from said distal end.

30-31. (canceled)

32. The device of claim 27, wherein:
said elongated catheter body comprises a main lumen passing from a proximal end of said elongated catheter body and terminating at a port proximal to and at a side of said distal end of said elongated catheter body;
said first-leaflet-engaging component is configured to move into and out of said main lumen through said port; and
said first leaflet-engaging component is functionally associated with a first leaflet-engaging component director, said first leaflet-engaging component director passing from said proximal end to said distal end of said elongated catheter body and through said main lumen of said elongated catheter body, said first leaflet-engaging component director is configured so that manipulation of said first leaflet-engaging component director affects said moving of said first leaflet-engaging component into and out of said main lumen through said port.

33. The device of claim 27, wherein said first leaflet-engaging component comprises a suction port configured for communication with a vacuum source through a vacuum lumen passing through said elongated catheter body.

34-37. (canceled)

38. The device of claim 32, further comprising:
a reversible anchoring component proximate to said distal end of said elongated catheter body, said anchoring component having at least two states:
i) a first non-anchoring state; and
ii) a second anchoring state, wherein said anchoring component engages walls of a coronary sinus in which said distal end is located so as stabilize a position of said distal end in said coronary sinus; and
an anchoring component actuator functionally associated with said anchoring component, configured to allow changing of said anchoring component from said first non-anchoring state to said second anchoring state.

39. (canceled)

40. The device of claim 38, in which the reversible anchoring component envelops said elongated catheter body, and comprises a side opening allowing a component to move into and out of said elongated catheter body through said port and through said side opening.

41. The device of claim 38, wherein said anchoring component is configured to avoid complete obstruction of a lumen of said coronary sinus when in said second anchoring state.

42-47. (canceled)

48. A kit comprising the device of claim 27, and a separate, additional leaflet augmentation device for augmenting at least one mitral valve leaflet.

49. A kit comprising the device of claim 27, and a separate, additional cardiac valve obstructor for obstructing at least part of a cardiac valve.

50. A device suitable for defining a minimally invasive conduit to a left atrium of a mammalian heart, comprising:
a) an elongated catheter body with a proximal end, a distal end and a distal tip, said distal end configured for passage into a coronary sinus from a peripheral region of a mammalian body;
b) a main lumen passing from said proximal end to said distal end of said elongated catheter body, said main lumen terminating at a side port located at a side of said distal end of said elongated catheter body;
c) a reversible anchoring component in proximity of said distal end of said elongated catheter body, said anchoring component having at least two states:
i) a first non-anchoring state; and
ii) a second anchoring state, wherein said anchoring component engages walls of a coronary sinus in which said distal end is located so as stabilize a position of said distal end in said coronary sinus; and
d) an anchoring component actuator functionally associated with said anchoring component, configured to allow changing of said anchoring component from said first non-anchoring state to said second anchoring state, wherein said anchoring component is configured to anchor on a side of said elongated catheter body opposite said side port.

51. The device of claim 50 and further comprising an elongated piercing component configured for passage through said main lumen from said proximal end to emerge out through said side port so as to puncture tissue proximate to said side port.

52. (canceled)

53. The device of claim 50, wherein said elongated catheter body is configured for passage of said distal end into a coronary sinus so that said side port faces a cardiac wall separating said coronary sinus from a left atrium.

54. The device of claim 53 in which said distal end is at an angle of between about 70° to 110° to a longitudinal axis of said elongated catheter body, said angle located proximally to said distal end, and in which said angle is located at about 5 centimeters +/−20% from said distal end.

55. (canceled)

56. A method for defining a minimally invasive conduit to a left atrium of a mammalian heart, comprising:
a) providing a device including:
i) an elongated catheter body with a proximal end, a distal end and a distal tip, said distal end configured for passage into a coronary sinus from a peripheral region of a mammalian body; and
ii) a main lumen passing from said proximal end to said distal end of said elongated catheter body, said main lumen terminating at a side port located at a side of said distal end of said elongated catheter body;
b) directing said distal end of said elongated catheter body of said device into a coronary sinus of a heart so that said side port faces a cardiac wall separating said coronary sinus from a left atrium; and
c) passing an elongated piercing component through said side port so as to puncture said cardiac wall separating said coronary sinus from a left atrium.

57-59. (canceled)

60. The method of claim 56, further comprising: pressing said side port against said cardiac wall separating said coronary sinus from said left atrium.

61-64. (canceled)

65. The method of claim 56 wherein said distal end of said elongated catheter body is configured to avoid complete obstruction of a lumen of said coronary sinus at least when in an anchoring state.
